SEQUENCE LISTING (43 sequences)

SEQ ID NO: 1 - protein, 31 aa, Artificial Sequence
  Synthetic
VWASRELERFAVNPGLLETSEGCRQILGQLQ

SEQ ID NO: 2 - protein, 212 aa, Artificial Sequence
  Synthetic
QAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGP

SEQ ID NO: 3 - protein, 36 aa, Artificial Sequence
  Synthetic
EALLDTGADDTVLEEMNLPGRWKPKMIGGIGGFIKV

SEQ ID NO: 4 - protein, 56 aa, Artificial Sequence
  Synthetic
TPDKKHQKEPPFLWMGYELHPDKWTVQPIVLPEKDSWTVNDIQKLVGKLNWASQIY

SEQ ID NO: 5 - protein, 31 aa, Artificial Sequence
  Synthetic
MENRWQVMIVWQVDRMRIRTWKSLVKHHMYI

SEQ ID NO: 6 - protein, 31 aa, Artificial Sequence
  Synthetic
DAKLVITTYWGLHTGERDWHLGQGVSIEWRK

SEQ ID NO: 7 - protein, 61 aa, Artificial Sequence
  Synthetic
FLGFLGAAGSTMGAASITLTVQARQLLSGIVQQQNNLLRAIEAQQHLLQLTVWGIKQLQAR

SEQ ID NO: 8 - protein, 31 aa, Artificial Sequence
  Synthetic
SLCLFSYHRLRDLLLIVTRIVELLGRRGWEA

SEQ ID NO: 9 - protein, 31 aa, Artificial Sequence
  Synthetic
NADCAWLEAQEEEEVGFPVRPQVPLRPMTYK

SEQ ID NO: 10 - protein, 31 aa, Artificial Sequence
  Synthetic
YSQKRQDILDLWVYHTQGYFPDWQNYTPGPG

SEQ ID NO: 11 - protein, 551 aa, Artificial Sequence
  Synthetic
VWASRELERFAVNPGLLETSEGCRQILGQLQQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPEALLDTGADDTVLEEMNLPGRWKPKMIGGIGGFIKVTPDKKHQKEPPFLWMGYELHPDKWTVQPIVLPEKDSWTVNDIQKLVGKLNWASQIYMENRWQVMIVWQVDRMRIRTWKSLVKHHMYIDAKLVITTYWGLHTGERDWHLGQGVSIEWRKFLGFLGAAGSTMGAASITLTVQARQLLSGIVQQQNNLLRAIEAQQHLLQLTVWGIKQLQARSLCLFSYHRLRDLLLIVTRIVELLGRRGWEANADCAWLEAQEEEEVGFPVRPQVPLRPMTYKYSQKRQDILDLWVYHTQGYFPDWQNYTPGPG

SEQ ID NO: 12 - protein, 551 aa, Artificial Sequence
  Synthetic
YSQKRQDILDLWVYHTQGYFPDWQNYTPGPGQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPTPDKKHQKEPPFLWMGYELHPDKWTVQPIVLPEKDSWTVNDIQKLVGKLNWASQIYDAKLVITTYWGLHTGERDWHLGQGVSIEWRKSLCLFSYHRLRDLLLIVTRIVELLGRRGWEAEALLDTGADDTVLEEMNLPGRWKPKMIGGIGGFIKVMENRWQVMIVWQVDRMRIRTWKSLVKHHMYIFLGFLGAAGSTMGAASITLTVQARQLLSGIVQQQNNLLRAIEAQQHLLQLTVWGIKQLQARNADCAWLEAQEEEEVGFPVRPQVPLRPMTYKVWASRELERFAVNPGLLETSEGCRQILGQLQ

SEQ ID NO: 13 - DNA, 1656 bp, Artificial Sequence
  Synthetic
atggtctgggccagcagagagctggaaagattcgccgtgaatcccggcctgctggaaacctctgagggctgcagacagatcctgggacagctgcagcaggccatctctcccagaacactgaacgcctgggtcaaagtggtggaagagaaggctttcagccccgaagtgatccccatgttcagcgccctttctgagggcgccacacctcaggacctgaacaccatgctgaataccgttggcggacaccaggccgccatgcagatgctgaaagagacaatcaacgaagaggccgccgagtgggatagactgcaccctgttcatgccggacctatcgctccaggccagatgagagagcctagaggctctgatatcgccggcaccaccagcacactgcaagagcagatcggctggatgaccaacaatcctcctattcctgtgggcgagatctacaagcggtggatcatcctgggcctgaacaagatcgtgcggatgtacagccccaccagcatcctggatatccggcagggacccaaagagcccttcagagactacgtggaccggttctacaagaccctgagagccgagcaggccagccaagaagtgaagaactggatgacagagacactgctggtgcagaacgccaatcctgactgcaagaccatcctgaaggccctgggacctgccgccacactggaagaaatgatgaccgcctgtcaaggcgttggcggccctgaagctttgctggatacaggcgccgatgacaccgtgctggaagagatgaatctgcctggccggtggaagcccaagatgatcggaggaatcggcggcttcatcaaagtgacccctgacaagaagcaccagaaagaaccacctttcctgtggatgggctacgagctgcaccccgataagtggaccgtgcagcctattgtgctgcccgagaaggatagctggaccgtgaacgacatccagaaactcgtgggcaagctgaattgggccagccagatctacatggaaaaccggtggcaagtgatgatcgtgtggcaggtcgaccggatgcggatcagaacctggaagtccctggtcaagcaccacatgtacatcgacgccaagctggtcatcaccacctactggggactgcacaccggcgagagagattggcatcttggacagggcgtgtcaatcgagtggcggaagttcctgggctttctgggagccgccggatctacaatgggagctgccagcatcaccctgacagtgcaggctagacagctgctgagcggaatcgtgcagcagcagaacaacctgctgagagccattgaggcccagcagcatctcctgcagctgacagtgtggggcatcaagcagctccaggctagaagcctgtgcctgttcagctaccacagactgagggacctgctgctgatcgtgacccggattgtggaactgctgggaagaagaggctgggaagccaatgccgattgcgcctggctggaagctcaagaggaagaggaagtcggcttccccgtcagacctcaggtgccactcagacccatgacctacaagtacagccagaagcggcaggacatcctggacctgtgggtgtaccacacacagggctacttccccgactggcagaactacacacctggaccaggc

SEQ ID NO: 14 - DNA, 1656 bp, Artificial Sequence
  Synthetic
atgtacagccagaagcggcaggacatcctggacctgtgggtgtaccacacacagggctacttccccgactggcagaactacacacctggaccaggacaggccatctctcccagaacactgaacgcctgggtcaaagtggtggaagagaaggctttcagccccgaagtgatccccatgttcagcgccctttctgagggcgccacacctcaggacctgaacaccatgctgaataccgttggcggacaccaggccgccatgcagatgctgaaagagacaatcaacgaagaggccgccgagtgggacagactgcatcctgttcatgccggacctatcgctcccggccagatgagagaacctagaggctctgatatcgccggcaccaccagcacactgcaagagcagatcggctggatgaccaacaatcctcctattcctgtgggcgagatctacaagcggtggatcatcctgggcctgaacaagatcgtgcggatgtactcccctaccagcatcctggatatccggcagggccccaaagagcccttcagagactacgtggaccggttctacaagaccctgagagccgagcaggccagccaagaagtgaagaactggatgacagagacactgctggtgcagaacgccaatcctgactgcaagaccatcctgaaggccctgggacctgccgccacactggaagaaatgatgaccgcctgtcaaggcgtcggcggacccacacctgataagaagcaccagaaagaaccaccgttcctgtggatgggctacgagctgcaccctgacaagtggaccgtgcagcctattgtgctgcccgagaaggatagctggaccgtgaacgacatccagaaactcgtgggcaagctgaactgggccagccagatctacgatgccaagctggtcatcaccacctactggggactgcacaccggcgagagagattggcatcttggacagggcgtgtccatcgagtggcggaagtccctgtgcctgttcagctaccacagactgagggacctgctgctgatcgtgacccggattgtggaactgctgggaagaagaggctgggaagccgaggctctgcttgatacaggcgccgatgataccgtgctggaagagatgaacctgcctggcagatggaagcccaagatgatcggcggcatcggcggattcatcaaagtcatggaaaaccggtggcaagtgatgatcgtgtggcaggtcgaccggatgcggatcagaacctggaagtctctggtcaagcaccacatgtatatctttctgggattcctgggcgctgccggctctacaatgggagccgcttctatcaccctgactgtgcaggctagacagctgctgagcggaatcgtgcagcagcagaacaacctgctgagagccattgaggcccagcagcatctcctgcagctgacagtgtggggcatcaagcagctccaggccagaaatgccgattgcgcctggctggaagctcaagaggaagaggaagtcggctttcccgtcagacctcaggtgccactgaggcctatgacctacaaagtgtgggccagcagagagctggaaagattcgccgtgaatcccggcctgctggaaacctctgagggctgcagacagatcctggggcagctgcag

SEQ ID NO: 15 - DNA, 93 bp, Artificial Sequence
  Synthetic
gtatgggcaagcagggagctagaacgattcgcagttaatcctggcctgttagaaacatcagaaggctgtagacaaatactgggacagctacaa

SEQ ID NO: 16 - DNA, 636 bp, Artificial Sequence
  Synthetic
caggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggaccc

SEQ ID NO: 17 - DNA, 108 bp, Artificial Sequence
  Synthetic
gaagctctattagatacaggagcagatgatacagtattagaagaaatgaatttgccaggaagatggaaaccaaaaatgatagggggaattggaggttttatcaaagta

SEQ ID NO: 18 - DNA, 168 bp, Artificial Sequence
  Synthetic
acaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttac

SEQ ID NO: 19 - DNA, 93 bp, Artificial Sequence
  Synthetic
atggaaaacagatggcaggtgatgattgtgtggcaagtagacaggatgaggattagaacatggaaaagtttagtaaaacaccatatgtatatt

SEQ ID NO: 20 - DNA, 93 bp, Artificial Sequence
  Synthetic
gatgctaaattggtaataacaacatattggggtctgcatacaggagaaagagactggcatttgggtcagggagtctccatagaatggaggaaa

SEQ ID NO: 21 - DNA, 183 bp, Artificial Sequence
  Synthetic
ttccttgggttcttgggagcagcaggaagcactatgggcgcagcctcaataacgctgacggtacaggccagacaattattgtctggtatagtgcagcagcagaacaatttgctgagggctattgaggcgcaacagcatctgttgcaactcacagtctggggcatcaagcagctccaggcaaga

SEQ ID NO: 22 - DNA, 93 bp, Artificial Sequence
  Synthetic
agcctgtgcctcttcagctaccaccgcttgagagacttactcttgattgtaacgaggattgtggaacttctgggacgcagggggtgggaagcc

SEQ ID NO: 23 - DNA, 93 bp, Artificial Sequence
  Synthetic
aatgctgattgtgcctggctagaagcacaagaggaggaggaggtgggttttccagtcagacctcaggtacctttaagaccaatgacttacaag

SEQ ID NO: 24 - DNA, 93 bp, Artificial Sequence
  Synthetic
tactcccaaaaaagacaagatatccttgatctgtgggtctaccacacacaaggctacttccctgattggcagaactacacaccagggccaggg

SEQ ID NO: 25 - protein, 500 aa, Artificial Sequence
  Synthetic
MGARASVLSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSKKKAQQAAADTGHSNQVSQNYPIVQNIQGQMVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKIVKCFNCGKEGHTARNCRAPRKKGCWKCGKEGHQMKDCTERQANFLGKIWPSYKGRPGNFLQSRPEPTAPPEESFRSGVETTTPPQKQEPIDKELYPLTSLRSLFGNDPSSQ

SEQ ID NO: 26 - protein, 1003 aa, Artificial Sequence
  Synthetic
FFREDLAFLQGKAREFSSEQTRANSPTRRELQVWGRDNNSPSEAGADRQGTVSFNFPQVTLWQRPLVTIKIGGQLKEALLDTGADDTVLEEMSLPGRWKPKMIGGIGGFIKVRQYDQILIEICGHKAIGTVLVGPTPVNIIGRNLLTQIGCTLNFPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVLFLDGIDKAQDEHEKYHSNWRAMASDFNLPPVVAKEIVASCDKCQLKGEAMHGQVDCSPGIWQLDCTHLEGKVILVAVHVASGYIEAEVIPAETGQETAYFLLKLAGRWPVKTIHTDNGSNFTGATVRAACWWAGIKQEFGIPYNPQSQGVVESMNKELKKIIGQVRDQAEHLKTAVQMAVFIHNFKRKGGIGGYSAGERIVDIIATDIQTKELQKQITKIQNFRVYYRDSRNPLWKGPAKLLWKGEGAVVIQDNSDIKVVPRRKAKIIRDYGKQMAGDDCVASRQDED

SEQ ID NO: 27 - protein, 856 aa, Artificial Sequence
  Synthetic
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 28 - protein, 192 aa, Artificial Sequence
  Synthetic
MENRWQVMIVWQVDRMRIRTWKSLVKHHMYISKKAKGWFYRHHYESTHPRISSEVHIPLGDAKLVITTYWGLHTGERDWHLGQGVSIEWRKRRYSTQVDPDLADQLIHLYYFDCFSESAIRNAILGHIVSPRCEYQAGHNKVGSLQYLALAALITPKKIKPPLPSVAKLTEDRWNKPQKTKGHRGSHTMNGH

SEQ ID NO: 29 - protein, 96 aa, Artificial Sequence
  Synthetic
MEQAPEDQGPQREPYNEWTLELLEELKNEAVRHFPRPWLHGLGQHIYETYGDTWAGVEAIIRILQQLLFIHFRIGCQHSRIGITRQRRARNGASRS

SEQ ID NO: 30 - protein, 101 aa, Artificial Sequence
  Synthetic
MEPVDPRLEPWKHPGSQPKTACTNCYCKKCCFHCQVCFITKGLGISYGRKKRRQRRRAPQDSQTHQVSLSKQPASQPRGDPTGPKESKKKVERETETDPVD

SEQ ID NO: 31 - protein, 116 aa, Artificial Sequence
  Synthetic
MAGRSGDSDEELLKTVRLIKFLYQSNPPPSPEGTRQARRNRRRRWRERQRQIRSISGWILSTYLGRPAEPVPLQLPPLERLTLDCSEDCGTSGTQGVGSPQILVESPAVLESGTKE

SEQ ID NO: 32 - protein, 78 aa, Artificial Sequence
  Synthetic
MQSLQILAIVALVVAAIIAIVVWSIVFIEYRKILRQRKIDRLIDRIRERAEDSGNESEGELSALVEMGHHAPWDVDDL

SEQ ID NO: 33 - protein, 206 aa, Artificial Sequence
  Synthetic
MGGKWSKRSVVGWPAVRERMRRAEPAADGVGAVSRDLEKHGAITSSNTAATNADCAWLEAQEEEEVGFPVRPQVPLRPMTYKGALDLSHFLKEKGGLEGLIYSQKRQDILDLWVYHTQGYFPDWQNYTPGPGIRYPLTFGWCFKLVPVEPEKVEEANEGENNCLLHPMSQHGMDDPEKEVLVWKFDSRLAFHHMARELHPEYYKDC

SEQ ID NO: 34 - protein, 458 aa, Artificial Sequence
  Synthetic
VWASRELERFAVNPGLLETSEGCRQILGQLQQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPEALLDTGADDTVLEEMNLPGRWKPKMIGGIGGFIKVTPDKKHQKEPPFLWMGYELHPDKWTVQPIVLPEKDSWTVNDIQKLVGKLNWASQIYDAKLVITTYWGLHTGERDWHLGQGVSIEWRKFLGFLGAAGSTMGAASITLTVQARQLLSGIVQQQNNLLRAIEAQQHLLQLTVWGIKQLQARSLCLFSYHRLRDLLLIVTRIVELLGRRGWEA

SEQ ID NO: 35 - protein, 458 aa, Artificial Sequence
  Synthetic
SLCLFSYHRLRDLLLIVTRIVELLGRRGWEAQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPTPDKKHQKEPPFLWMGYELHPDKWTVQPIVLPEKDSWTVNDIQKLVGKLNWASQIYFLGFLGAAGSTMGAASITLTVQARQLLSGIVQQQNNLLRAIEAQQHLLQLTVWGIKQLQAREALLDTGADDTVLEEMNLPGRWKPKMIGGIGGFIKVDAKLVITTYWGLHTGERDWHLGQGVSIEWRKVWASRELERFAVNPGLLETSEGCRQILGQLQ

SEQ ID NO: 36 - DNA, 1374 bp, Artificial Sequence
  Synthetic
gtatgggcaagcagggagctagaacgattcgcagttaatcctggcctgttagaaacatcagaaggctgtagacaaatactgggacagctacaacaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccgaagctctattagatacaggagcagatgatacagtattagaagaaatgaatttgccaggaagatggaaaccaaaaatgatagggggaattggaggttttatcaaagtaacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacgatgctaaattggtaataacaacatattggggtctgcatacaggagaaagagactggcatttgggtcagggagtctccatagaatggaggaaattccttgggttcttgggagcagcaggaagcactatgggcgcagcctcaataacgctgacggtacaggccagacaattattgtctggtatagtgcagcagcagaacaatttgctgagggctattgaggcgcaacagcatctgttgcaactcacagtctggggcatcaagcagctccaggcaagaagcctgtgcctcttcagctaccaccgcttgagagacttactcttgattgtaacgaggattgtggaacttctgggacgcagggggtgggaagcc

SEQ ID NO: 37 - DNA, 1374 bp, Artificial Sequence
  Synthetic
agcctgtgcctcttcagctaccaccgcttgagagacttactcttgattgtaacgaggattgtggaacttctgggacgcagggggtgggaagcccaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacttccttgggttcttgggagcagcaggaagcactatgggcgcagcctcaataacgctgacggtacaggccagacaattattgtctggtatagtgcagcagcagaacaatttgctgagggctattgaggcgcaacagcatctgttgcaactcacagtctggggcatcaagcagctccaggcaagagaagctctattagatacaggagcagatgatacagtattagaagaaatgaatttgccaggaagatggaaaccaaaaatgatagggggaattggaggttttatcaaagtagatgctaaattggtaataacaacatattggggtctgcatacaggagaaagagactggcatttgggtcagggagtctccatagaatggaggaaagtatgggcaagcagggagctagaacgattcgcagttaatcctggcctgttagaaacatcagaaggctgtagacaaatactgggacagctacaa

SEQ ID NO: 38 - DNA, 1653 bp, Artificial Sequence
  Synthetic
gtatgggcaagcagggagctagaacgattcgcagttaatcctggcctgttagaaacatcagaaggctgtagacaaatactgggacagctacaacaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccgaagctctattagatacaggagcagatgatacagtattagaagaaatgaatttgccaggaagatggaaaccaaaaatgatagggggaattggaggttttatcaaagtaacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacatggaaaacagatggcaggtgatgattgtgtggcaagtagacaggatgaggattagaacatggaaaagtttagtaaaacaccatatgtatattgatgctaaattggtaataacaacatattggggtctgcatacaggagaaagagactggcatttgggtcagggagtctccatagaatggaggaaattccttgggttcttgggagcagcaggaagcactatgggcgcagcctcaataacgctgacggtacaggccagacaattattgtctggtatagtgcagcagcagaacaatttgctgagggctattgaggcgcaacagcatctgttgcaactcacagtctggggcatcaagcagctccaggcaagaagcctgtgcctcttcagctaccaccgcttgagagacttactcttgattgtaacgaggattgtggaacttctgggacgcagggggtgggaagccaatgctgattgtgcctggctagaagcacaagaggaggaggaggtgggttttccagtcagacctcaggtacctttaagaccaatgacttacaagtactcccaaaaaagacaagatatccttgatctgtgggtctaccacacacaaggctacttccctgattggcagaactacacaccagggccaggg

SEQ ID NO: 39 - DNA, 1653 bp, Artificial Sequence
  Synthetic
tactcccaaaaaagacaagatatccttgatctgtgggtctaccacacacaaggctacttccctgattggcagaactacacaccagggccagggcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcaggaacaaataggatggatgacaaataatccacctatcccagtaggagaaatttataaaagatggataatcctgggattaaataaaatagtaagaatgtatagccctaccagcattctggacataagacaaggaccaaaggaaccctttagagactatgtagaccggttctataaaactctaagagccgagcaagcttcacaggaggtaaaaaattggatgacagaaaccttgttggtccaaaatgcgaacccagattgtaagactattttaaaagcattgggaccagcggctacactagaagaaatgatgacagcatgtcagggagtaggaggacccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacgatgctaaattggtaataacaacatattggggtctgcatacaggagaaagagactggcatttgggtcagggagtctccatagaatggaggaaaagcctgtgcctcttcagctaccaccgcttgagagacttactcttgattgtaacgaggattgtggaacttctgggacgcagggggtgggaagccgaagctctattagatacaggagcagatgatacagtattagaagaaatgaatttgccaggaagatggaaaccaaaaatgatagggggaattggaggttttatcaaagtaatggaaaacagatggcaggtgatgattgtgtggcaagtagacaggatgaggattagaacatggaaaagtttagtaaaacaccatatgtatattttccttgggttcttgggagcagcaggaagcactatgggcgcagcctcaataacgctgacggtacaggccagacaattattgtctggtatagtgcagcagcagaacaatttgctgagggctattgaggcgcaacagcatctgttgcaactcacagtctggggcatcaagcagctccaggcaagaaatgctgattgtgcctggctagaagcacaagaggaggaggaggtgggttttccagtcagacctcaggtacctttaagaccaatgacttacaaggtatgggcaagcagggagctagaacgattcgcagttaatcctggcctgttagaaacatcagaaggctgtagacaaatactgggacagctacaa

SEQ ID NO: 40 - protein, 552 aa, Artificial Sequence
  Synthetic
MVWASRELERFAVNPGLLETSEGCRQILGQLQQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPEALLDTGADDTVLEEMNLPGRWKPKMIGGIGGFIKVTPDKKHQKEPPFLWMGYELHPDKWTVQPIVLPEKDSWTVNDIQKLVGKLNWASQIYMENRWQVMIVWQVDRMRIRTWKSLVKHHMYIDAKLVITTYWGLHTGERDWHLGQGVSIEWRKFLGFLGAAGSTMGAASITLTVQARQLLSGIVQQQNNLLRAIEAQQHLLQLTVWGIKQLQARSLCLFSYHRLRDLLLIVTRIVELLGRRGWEANADCAWLEAQEEEEVGFPVRPQVPLRPMTYKYSQKRQDILDLWVYHTQGYFPDWQNYTPGPG

SEQ ID NO: 41 - protein, 552 aa, Artificial Sequence
  Synthetic
MYSQKRQDILDLWVYHTQGYFPDWQNYTPGPGQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPTPDKKHQKEPPFLWMGYELHPDKWTVQPIVLPEKDSWTVNDIQKLVGKLNWASQIYDAKLVITTYWGLHTGERDWHLGQGVSIEWRKSLCLFSYHRLRDLLLIVTRIVELLGRRGWEAEALLDTGADDTVLEEMNLPGRWKPKMIGGIGGFIKVMENRWQVMIVWQVDRMRIRTWKSLVKHHMYIFLGFLGAAGSTMGAASITLTVQARQLLSGIVQQQNNLLRAIEAQQHLLQLTVWGIKQLQARNADCAWLEAQEEEEVGFPVRPQVPLRPMTYKVWASRELERFAVNPGLLETSEGCRQILGQLQ

SEQ ID NO: 42 - protein, 459 aa, Artificial Sequence
  Synthetic
MVWASRELERFAVNPGLLETSEGCRQILGQLQQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPEALLDTGADDTVLEEMNLPGRWKPKMIGGIGGFIKVTPDKKHQKEPPFLWMGYELHPDKWTVQPIVLPEKDSWTVNDIQKLVGKLNWASQIYDAKLVITTYWGLHTGERDWHLGQGVSIEWRKFLGFLGAAGSTMGAASITLTVQARQLLSGIVQQQNNLLRAIEAQQHLLQLTVWGIKQLQARSLCLFSYHRLRDLLLIVTRIVELLGRRGWEA

SEQ ID NO: 43 - protein, 459 aa, Artificial Sequence
  Synthetic
MSLCLFSYHRLRDLLLIVTRIVELLGRRGWEAQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPTPDKKHQKEPPFLWMGYELHPDKWTVQPIVLPEKDSWTVNDIQKLVGKLNWASQIYFLGFLGAAGSTMGAASITLTVQARQLLSGIVQQQNNLLRAIEAQQHLLQLTVWGIKQLQAREALLDTGADDTVLEEMNLPGRWKPKMIGGIGGFIKVDAKLVITTYWGLHTGERDWHLGQGVSIEWRKVWASRELERFAVNPGLLETSEGCRQILGQLQ

(19) United States
(12) Patent Application Publication
(10) Pub. No.: US 2021/0317192 A9
(43) Pub. Date: Oct. 14, 2021

(54) HIV-1 SPECIFIC IMMUNOGEN COMPOSITIONS AND METHODS OF USE

(71) Applicants: Massachusetts Institute of Technology, Cambridge, MA (US); The General Hospital Corporation, Boston, MA (US); Beth Israel Deaconess Medical Center, Inc., Boston, MA (US)

(72) Inventors: Darrell J. Irvine, Arlington, MA (US); Dan H. Barouch, Newton, MA (US); Arup K. Chakraborty, Lexington, MA (US); Dariusz Murakowski, Cambridge, MA (US); Bruce D. Walker, Cambridge, MA (US); John Barton, Riverside, CA (US); Andrew Ferguson, Chicago, IL (US)

(73) Assignees: Massachusetts Institute of Technology, Cambridge, MA (US); The General Hospital Corporation, Boston, MA (US); Beth Israel Deaconess Medical Center, Inc., Boston, MA (US)

(21) Appl. No.: 16/887,710

(22) Filed: May 29, 2020

Related U.S. Application Data
(60) Provisional application No. 62/853,919, filed on May 29, 2019.

(51) Int. Cl. C07K 16/10; A61K 39/12; A61P 31/14; C12N 15/86

(52) U.S. Cl. CPC C07K 16/1045; A61K 39/12; G16B 50/30; C12N 15/86; A61P 31/14

(57) ABSTRACT

Disclosed herein are methods and compositions for treating a subject having or at risk of having an HIV infection. Disclosed herein are peptide immunogens and nucleic acids that have epitopes in which mutations are most likely to have deleterious effects on the HIV virus. An algorithm is disclosed for the selection of the epitopes based on the HIV fitness landscape, and it accounts for the effect of coupling mutations.

### RELATED APPLICATIONS

This application claims the benefit under 35 U.S.C. § 119(e) of U.S. Provisional Application Ser. No. 62/853,919 entitled “HIV-1 SPECIFIC IMMUNOGEN COMPOSITIONS AND METHODS OF USE” filed on May 29, 2019, the entire contents of which are incorporated by reference herein.

### BACKGROUND OF THE INVENTION

The human immunodeficiency virus (HIV) is transmitted through certain body fluids (e.g. blood, semen). The virus targets and destroys the body's immune system, specifically targeting CD4 cells (also referred to as T cells). Over time, this process can leave an infected individual severely immunocompromised and vulnerable to secondary infections (e.g. opportunistic infections). The compromised immune system also increases the severity of these secondary infections. Examples of opportunistic infections include Herpes simplex virus 1 (HSV-1) infection, pneumonia, Salmonella infection, candidiasis (thrush), toxoplasmosis, Toxoplasmosis, and tuberculosis (TB).

The three stages of HIV infection are: (1) acute HIV infection, (2) clinical latency, and (3) AIDS (acquired immunodeficiency syndrome). The acute HIV infection is approximately 2-4 weeks following infection and is characterized by high viral load. Individuals in this stage exhibit flu-like symptoms. There is high risk of transmission during this stage. The clinical latency stage is the asymptomatic stage, wherein viral reproduction is at a low rate. The AIDS stage occurs when the CD4 cell count has drastically declined (e.g. below 200 cells/mm³) and/or the infected individual develops an opportunistic infection.

HIV infection is currently treated using antiretroviral therapy (ART). Effective treatment is achieved through early detection and daily treatment. If administered early and on a daily basis, ART can prolong the life of a patient, in some cases, keeping the HIV infection in the clinical latency phase for about a decade. Historically, vaccination has been the best method for preventing infectious disease. However, previous attempts to develop a safe and effective vaccine for HIV have been unsuccessful.

### SUMMARY OF THE INVENTION

The present disclosure is based, at least in part, on methods and compositions for treating a subject having or at risk of having an HIV (e.g., HIV-1) infection. The present disclosure provides peptide immunogens, which may be referred to herein as multiunit immunogens, and nucleic acids encoding such immunogens. The peptide immunogens comprise epitopes from HIV-1 proteome that are especially vulnerable to mutations in diverse sequence backgrounds. These peptide immunogens and the nucleic acids that encode such immunogens may be used to stimulate anti-HIV-1 immune responses in subjects, thereby providing in such subjects immunity against HIV-1. Thus, in some instances, these proteins and their encoding nucleic acids may serve as a vaccine for HIV-1.

Accordingly, one aspect of the present disclosure provides a peptide immunogen comprising a plurality of HIV-1-specific immunogen subunits each having an amino acid sequence selected from the group consisting of SEQ ID NO: 1, 2, 3, 4, 5, 6, 7, 8, 9, and 10, or any combination thereof and in any order. These immunogen subunits are provided in Table 1 and may be referred to herein by their SEQ ID NO: or may be simply referred to as subunit 1 (corresponding to SEQ ID NO:1), subunit 2 (corresponding to SEQ ID NO:2), and so on. In some embodiments, the plurality of HIV-1 specific immunogen subunits is 5, 6, 7, 8, 9 or 10 HIV-1 specific immunogen subunits, in any order. In some embodiments, the peptide immunogen comprises any order of 5 or more of the HIV-1-specific immunogen subunits. In some embodiments, the peptide immunogen has an amino acid sequence of:

B₁B₂B₃B₄B₅B₆B₇B₈B₉B₁₀

wherein B₁, B₂, B₃, B₄, B₅, B₆, B₇, B₈, B₉, and B₁₀ are SEQ ID NOs: 1, 2, 3, 4, 5, 6, 7, 8, 9, and 10, respectively. In some embodiments, the peptide immunogen has an amino acid sequence of SEQ ID NO:11 or SEQ ID NO:40, which represents a peptide immunogen comprising in order subunits 1-10 represented by SEQ ID NOs: 1, 2, 3, 4, 5, 6, 7, 8, 9 and 10 respectively. In some embodiments, the amino acid sequence is SEQ ID NO:12 or SEQ ID NO:41, which represents a shuffled form of the peptide immunogen, comprising in order subunits 10, 2, 4, 6, 8, 3, 5, 7, 9, and 1 represented by SEQ ID NOs: 10, 2, 4, 6, 8, 3, 5, 7, 9, and 1 respectively.

In some embodiments, the peptide immunogen has fewer than ten subunits. As an example, the peptide immunogen may have an amino acid sequence of:

B₁B₂B₃B₄B₆B₇B₈

wherein B₁, B₂, B₃, B₄, B₆, B₇, and B₈ are SEQ ID NOs: 1, 2, 3, 4, 6, 7, and 8 respectively. In some embodiments, the amino acid sequence is SEQ ID NO:34 or SEQ ID NO:42, which represents a peptide immunogen, comprising in order subunits 1, 2, 3, 4, 6, 7, and 8 represented by SEQ ID NOs: 1, 2, 3, 4, 6, 7, and 8 respectively. In some embodiments, the amino acid sequence is SEQ ID NO:35 or SEQ ID NO:43, which represents a shuffled form of the shorter peptide immunogen, comprising in order subunits 8, 2, 4, 7, 3, 6, and 1 represented by SEQ ID NOs: 8, 2, 4, 7, 3, 6, and 1 respectively.

It will be understood by those in the art that any transcribed protein will typically begin with a methionine residue. Thus the disclosure contemplates and embraces all peptide immunogen amino acid sequences provided herein with a methionine in the first position. Similarly, the disclosure contemplates and embraces all nucleotide sequences encoding such peptide immunogens with a start codon (e.g., ATG or AUG) in the first codon position.

In some embodiments, conjugation of each HIV-1-specific immunogen subunit to another HIV-1 specific immunogen subunit creates a junctional epitope, wherein each junctional epitope is present once in the peptide immunogen. In some embodiments, one or more of the HIV-1-specific immunogen subunits is repeated, optionally repeated once, provided that the repeated subunits are flanked by different subunits relative to each other, thereby creating different junctional epitopes at each repeated subunit. In some embodiments, the length of the peptide immunogen ranges from 300 to 1,600 residues.

Another aspect of the present disclosure provides a nucleic acid comprising a nucleotide sequence that encodes any one of the peptide immunogens herein. The nucleic acid may comprise any number and any combination of immunogen subunit coding (nucleotide) sequences selected from the group consisting of SEQ ID NO: 15, 16, 17, 18, 19, 20, 21, 22, 23, and 24, which encode the amino acid sequences of SEQ ID NOs: 1, 2, 3, 4, 5, 6, 7, 8, 9, and 10 respectively which in turn represent subunits 1-10. In some embodiments, the nucleotide sequence is SEQ ID NO:13 or SEQ ID NO:38, which encodes the immunogen having amino acid sequence of SEQ ID NO:40 or SEQ ID NO:11. In some embodiments, the nucleotide sequence is SEQ ID NO:14 or SEQ ID NO:39, which encodes the immunogen having amino acid sequence of SEQ ID NO:41 or SEQ ID NO:12. In some embodiments, the nucleotide sequence is SEQ ID NO:36, which encodes the immunogen having amino acid sequence of SEQ ID NO:34 and with an additional start codon will encode SEQ ID NO:42. In some embodiments, the nucleotide sequence is SEQ ID NO:37, which encodes the immunogen having amino acid sequence of SEQ ID NO:35 and with an additional start codon will encode SEQ ID NO:43.

As will be understood in the art, due to the degeneracy of the genetic code (or codons), other nucleotide sequences may also encode the various amino acid sequences provided herein and these nucleotide sequences will be readily apparent based on the amino acid sequences provided herein. The disclosure further contemplates nucleotide sequences that comprise a start codon in the first position, as is shown in SEQ ID NO:13. SEQ ID NO: 38 similarly may be used with a start codon in the first codon position. Similar teachings apply to SEQ ID NOs: 14 and 39.

In some embodiments, the nucleic acid is a nucleic acid vector. In some embodiments, the nucleic acid vector is a DNA vector. In some embodiments, the nucleic acid vector is an RNA vector. In some embodiments, the nucleic acid vector is a viral vector. In some embodiments, the nucleic acid vector is an adenoviral vector. In some embodiments, the nucleic acid vector is an adenovirus-associated viral vector. In some embodiments, the nucleic acid vector is a replication incompetent adenovirus vector. In some embodiments, the nucleic acid vector is derived from a human serotype selected from the group consisting of Ad5, Ad11, Ad35, Ad50, Ad26, Ad48, and Ad49. In some embodiments, the nucleic acid vector is derived from a rhesus adenovirus vector. In some embodiments, the rhesus adenovirus vector is RhAd51, RhAd52 or RhAd53.

Another aspect of the present disclosure composition comprising a peptide immunogen of as disclosed herein. In some embodiments, the composition is a pharmaceutical composition. In some embodiments, the composition further comprises an adjuvant. In some embodiments, the adjuvant is an alum-based adjuvant. In some embodiments, the composition is formulated for intramuscular injection. In some embodiments, the composition comprises a nucleic acid as disclosed herein. In some embodiments, the composition is a pharmaceutical composition. In some embodiments, the composition is formulated for intramuscular injection.

Another aspect of the present disclosure provides a method for treating a subject having or at risk of having an HIV-1 infection, comprising administering to said subject an effective amount of a peptide immunogen as described herein. In some embodiments, the subject is administered a prime dose and a boost dose of the peptide immunogen. In some embodiments, the peptide immunogens of the prime dose and the boost dose are different from each other. In some embodiments, the subject is a subject having an HIV-1 infection. In some embodiments, the subject is a subject at risk of having an HIV-1 infection. In some embodiments, the subject has AIDS. In some embodiments, the method further comprises administering an anti-viral agent to the subject.

Another aspect of the present disclosure provides a method, comprising accessing viral fitness information associated with one or more proteins of a virus and at least one protein sequence corresponding to the one or more proteins; determining, using the viral fitness information, a combination of epitopes occurring in the at least one protein sequence as having a high fitness cost; and generating an output indicating subunits of the at least one protein sequence that have sequences of the epitopes in the combination. In some embodiments, the combination of epitopes includes epitopes that account for coupling mutations of the at least one protein sequence. In some embodiments, the combination of epitopes includes one or more deleterious mutation regions of the at least one protein sequence. In some embodiments, the virus is HIV. In some embodiments, determining the combination of epitopes further comprises determining a first pair of epitopes as having a high fitness cost; comparing a fitness cost for a set of epitopes that includes the first pair and at least one other epitope to a first threshold value; and determining the combination of epitopes based at least in part of the comparing. In some embodiments, determining the combination of epitopes further comprises including the first pair of epitopes and the at least one other epitope in the combination if the fitness cost is above the first threshold value. In some embodiments, determining the combination of epitopes further comprises including the first pair of epitopes in the combination if the fitness cost is below the first threshold value. In some embodiments, generating the output indicating subunits further comprises determining one or more residues of the at least one protein to include in the subunits that exists outside the combination of epitopes. In some embodiments, generating the output indicating subunits further comprises determining at least one of the epitopes to exclude from the subunits. In some embodiments, the method further comprises generating a polypeptide sequence for an immunogen having the combination of epitopes. In some embodiments, the method further comprises generating a nucleic acid sequence for a vector that encodes for the immunogen. In some embodiments, the vector is an adenoviral vector, and the immunogen has a length between 300 to 1600 residues.

Another aspect of the present disclosure provides a system comprising at least one hardware processor; and at least one non-transitory computer-readable storage medium storing processor-executable instructions that, when executed by the at least one hardware processor, cause the at least one hardware processor to perform the methods disclosed herein.

Another aspect of the present disclosure provides at least one non-transitory computer-readable storage medium storing processor-executable instructions that, when executed by at least one hardware processor, cause the at least one hardware processor to perform the methods disclosed herein.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. For purposes of clarity, not every component may be labeled in every drawing. It is to be understood that the data illustrated in the drawings in no way limit the scope of the disclosure. The color versions of these Figures are available in the file wrapper of U.S. Provisional Application No. 62/853,919 filed May 29, 2019, to which priority is claimed. In the drawings:

FIG. 1 includes a schematic of the immunogen design algorithm for the adenovirus delivery platform.

FIG. 2 includes diagrams showing the sequence coverage and number of subunits as a function of threshold values E1 and E2. Since subunits may overlap, the sequence coverage underestimates the total length of the immunogen.

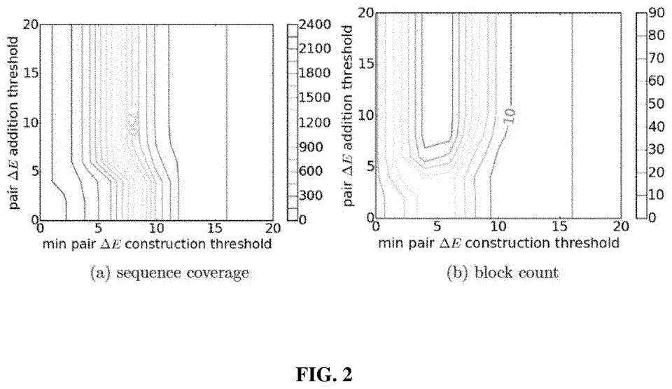

FIG. 3 shows the subunits selected in the gag protein of HIV-1, which are underlined and correspond to SEQ ID NOs: 1 and 2. The Figure further provides the amino acid sequence of the gag protein (SEQ ID NO:25).

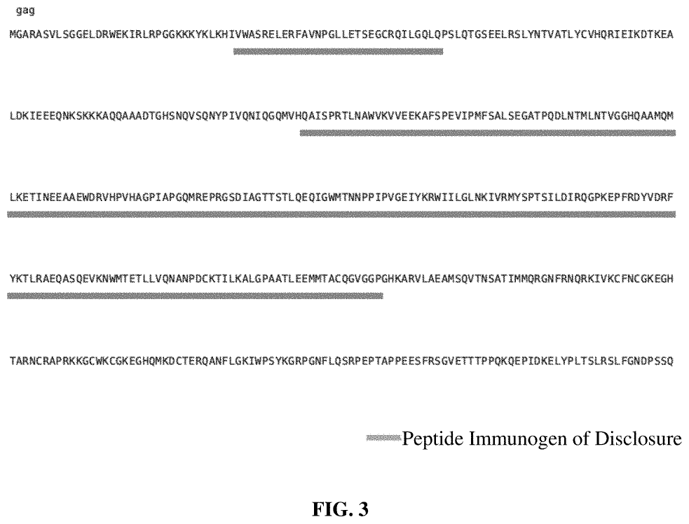

FIG. 4 shows the subunits selected in the pol protein of HIV-1, which are underlined and correspond to SEQ ID NOs: 3 and 4. The Figure further provides the amino acid sequence of the pol protein (SEQ ID NO:26).

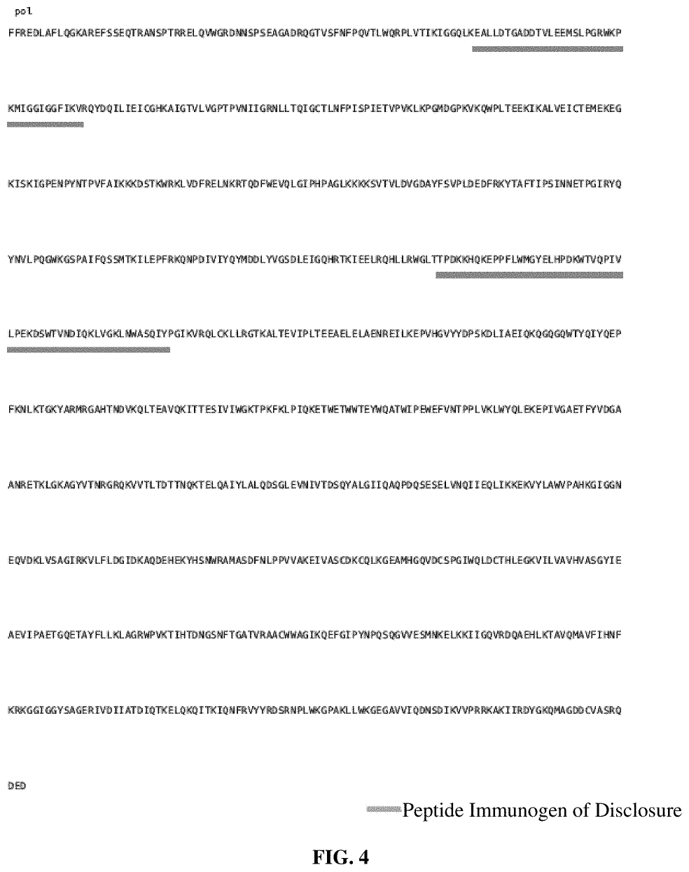

FIG. 5 shows the subunits selected in the env protein of HIV-1, which are underlined and correspond to SEQ ID NOs: 7 and 8. The Figure further provides the amino acid sequence of the env protein (SEQ ID NO:27).

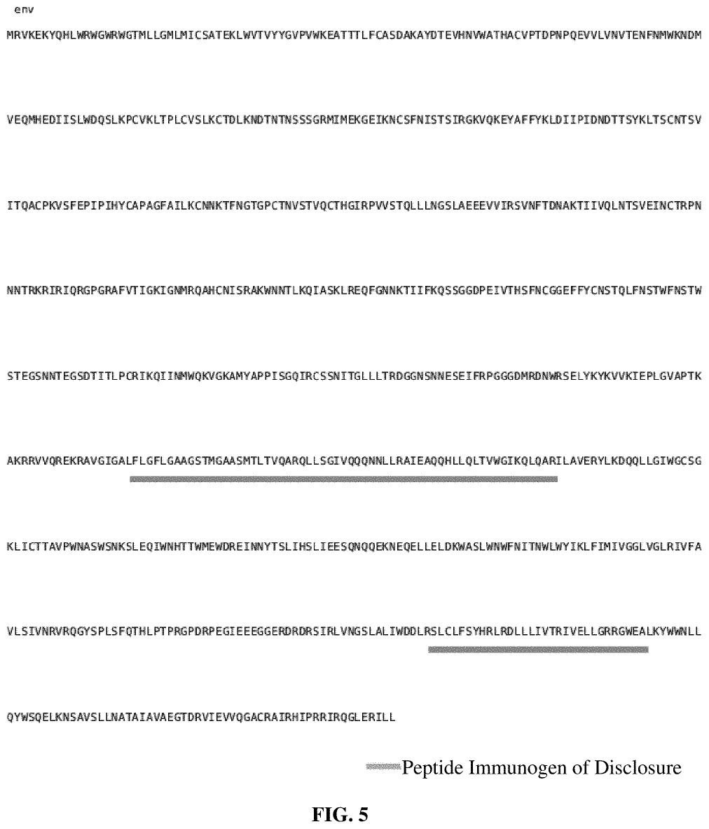

FIG. 6 shows the subunits selected in the vif, and nef proteins of HIV-1, which are underlined and correspond to SEQ ID NOs: 5, 6, 9 and 10. The Figure further provides the amino acid sequences of the vif, vpr, tat, rev, vpu, and nef proteins (SEQ ID NO:28-33).

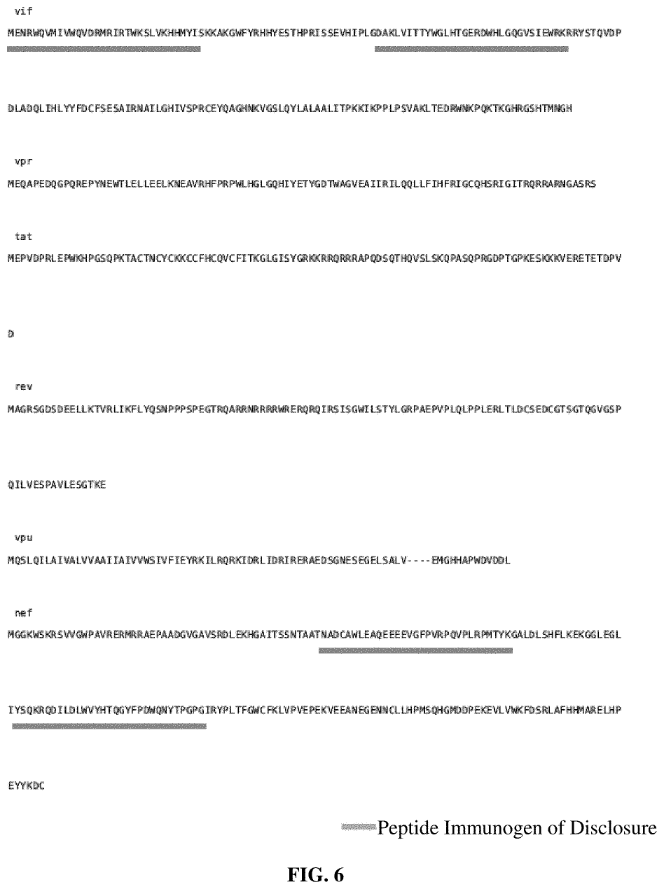

FIGS. 7A and 7B are bar graphs showing immunogenicity to various peptide pools for Macaques at 4 weeks after priming (FIG. 7A) and at 50 weeks after boosting (FIG. 7B). The first 4 Macaques (12-041, 12-056, 12-077, 12-120) were immunized with the immunogen in the present disclosure. The immunogen for the prime was the shuffled immunogen (amino acid SEQ ID NO: 12 and with an M inserted in the first position, encoded by nucleotide sequence SEQ ID NO:14) in the present disclosure, and it was vectored by Adenovirus serotype 26. The later boost used the other immunogen (amino acid SEQ ID NO:11 with an M inserted in the first position, encoded by nucleotide sequence SEQ ID NO:13) in the present disclosure, and it was vectored by Adenovirus serotype 5. The last two Macaques (12-158 and 12-172) were immunized with standard whole protein immunogens with Adenovirus serotype 26 vector for the prime and Adenovirus serotype 5HVR48 (99% identical to Adenovirus serotype 5) for the boost. Immunogenicity was measured for three different peptide pools (PET, Mos 1, and Mos 2) using standard ELISPOT assays, and the results are reported as the number of spot forming cells (SFC) per million peripheral blood mononuclear cells (PBMC).

FIG. 8 is a diagram of an illustrative processing pipeline for designing immunogens, in accordance with some embodiments of the technology described herein.

FIG. 9 is a flow chart of an illustrative process for designing immunogens, in accordance with some embodiments of the technology described herein.

FIG. 10 is a block diagram of an illustrative computer system that may be used in implementing some embodiments of the technology described herein.

### DETAILED DESCRIPTION OF THE INVENTION

The present disclosure provides, in part, novel peptide immunogens comprising a plurality of epitopes from the HIV-1 proteome and nucleic acids encoding such immunogens. These epitopes are selected based on the fitness cost of mutations in the epitopes and accounts for coupling of mutations. These peptide immunogens and nucleic acids are useful for the treatment of a subject having or at risk of developing an HIV (e.g., HIV-1) infection. This disclosure therefore provides compositions comprising such peptide immunogens or their encoding nucleic acids, and such compositions may be used therapeutically or prophylactically. The immunogens may be administered in a single dose or in a plurality of doses (e.g., a prime dose followed by one or more boost doses). As described in greater detail herein, the immunogens contained in such doses may be identical or they may be different from each other. In some embodiments, the peptide immunogens or nucleic acids in the prime and boost doses are different, thereby minimizing the unintended effects of junctional epitopes (as described herein) in the peptide immunogen.

### I. Immunogen and Treatment

### Peptide Immunogen

This disclosure provides, in part, novel and robust peptide immunogens for inducing anti-HIV (e.g., HIV-1) immune responses in vivo. This disclosure provides a number of examples of such immunogens, as well as the methodology for creating such immunogens from HIV and other pathogens. The peptide immunogens provided herein were made using fitness landscapes for the HIV proteome. Provided herein is an algorithm that uses fitness landscape metrics to arrive at peptide immunogens that are more robust and less susceptible to HIV mutation strategy than immunogens prepared heretofore. These immunogens comprise select regions of the HIV-1 proteome. Such regions, referred to herein as immunogen subunits, are derived from different proteins of the HIV-1 proteome. The immunogens are concatamers of these subunits, and therefore comprise subunits from two or more proteins connected to each other, in any order. In accordance with this disclosure, the peptide immunogens include regions where mutations are especially deleterious in all possible viral protein sequence backgrounds and importantly exclude regions within the HIV-1 proteome that are rife with compensatory mutations. Thus, these peptide immunogens are modular (multi-unit) constructs, comprised of subunits that have been determined to have the most deleterious effects on HIV viral fitness in diverse sequence backgrounds.

“Viral fitness” is a parameter that may be defined as the replicative adaptation of an organism to its environment. Mutations (e.g. single amino acid mutations) can reduce viral fitness, but this effect may be countered by compensatory mutations. In the case of certain viruses, e.g. HIV, fitter viruses may be considered to be more prevalent. An assumption that the rank order of prevalence is statistically similar to the rank order of the intrinsic fitness in viruses such as HIV-1 allows the use of prevalence data (the prevalence landscape) to infer the fitness landscape (Barton et al., Nature Communications, 2015). By applying the algorithm disclosed herein in combination with an HIV-1 fitness landscape, HIV-1 proteome subunits are identified and then concatenated to make a peptide immunogen that can be used in vivo or ex vivo to stimulate an anti-HIV-1 immune response in a subject for prophylactic or therapeutic treatment.

As used herein, the term “subunit” refers to an amino acid sequence comprising at least one epitope, wherein the amino acid sequence is at least 31 residues in length. These 31 residues are contiguous residues in the HIV-1 proteome. As used herein, the term “epitope” refers to an amino acid sequence that is 11 residues in length. These 11 residues are contiguous residues in the HIV-1 proteome. The epitope may be referred to herein as an 11-mer epitope.

The subunits in the disclosed peptide immunogens comprise one or more epitopes that are selected based on the expected fitness cost of mutations. The “fitness cost” of a mutation is indicative of the deleterious effect said mutation may have on the viral fitness. For example, if the inclusion of an epitope in an immunogen elicits an immune response that a virus (e.g. HIV) can evade (escape) by compensatory mutations elsewhere in the viral genome, the epitope is said to have a low fitness cost, and the more compensatory mutations present in the viral genome, the lower the epitope's fitness cost. In contrast, an epitope having a higher fitness cost, if mutated, would have a more deleterious effect on the virus. In some cases where fitness cost of an epitope is high (relative to other epitopes in the proteome), the virus would be unable to evade the immune response to that epitope and survive. The fitness cost accounts for the epistatic interactions and potential escape mutations in various sequence backgrounds. As used, the term “sequence background” refers to the residues that are within a protein but outside of an epitope of interest.

Regions of HIV proteins where mutations are most likely to be deleterious in diverse sequence backgrounds can be widely interspaced. Therefore, selecting long, contiguous regions of the desired length that also maximize the expected fitness cost of mutations in diverse sequence backgrounds is a challenge. This disclosure addresses that challenge by providing an immunogen that consists of discrete subunits that contain the most vulnerable regions, regardless of whether such subunits are contiguously located in the naturally occurring viral proteome. These subunits are then concatenated to obtain an immunogen with the overall desired length. As used herein, the terms “concatenation” and “conjugation” are used interchangeably and refer to the covalent linkage of two distinct subunits by a peptide linkage (in case of peptide immunogens) or a phosphodiester linkage (in some cases of nucleic acids). The subunits are typically physically separated in the naturally occurring HIV-1 proteome (i.e., they are not adjacent to each other but are instead separated from each other by 1 or more amino acid residues, including for example 5, 10, 15, 20, 50, etc. amino acid residues.

Concatenation of these subunits creates regions which are not naturally occurring and which when presented in a subject may cause an immune response in the subject. Such immune response however is not useful as it is directed to the immunogen but not the HIV-1 virus. Accordingly, the immunogens provided herein are designed to limit the effect of these “junctional epitopes”. As used, the term “junctional epitopes” refers to non-naturally occurring epitopes that occur in a sequence as a result of the conjunction of subunits that are not adjacent in the naturally occurring HIV proteome. The probability of inducing an immune response against a junctional epitope is reduced by reducing the number of junctional epitopes in an immunogen. This may be accomplished in part by controlling the minimum length of the subunits. Therefore, the subunits of the present disclosure are at least 31 residues in length. This number represents the minimum length at which the number of true epitopes (i.e., those present in the HIV-1 proteome) exceeds the number of junctional epitopes.

The peptide immunogens of the present disclosure comprise subunits from two or more distinct HIV-1 proteins. Table 1 shows the subunits that can be used in the peptide immunogens of the present disclosure. The subunits within the immunogens of the present disclosure can be rearranged (of shuffled) to make various peptide immunogens. All different combinations and permutations of the subunits in Table 1 are contemplated. For example, the immunogen may comprise any 2, any 3, any 4, any 5, any 6, any 7, any 8, any 9, or all 10 of the subunits in Table 1, in any order. The immunogen may comprise one or more subunits from 2, 3, 4 or 5 HIV-1 proteins.

TABLE 1
The subunits that can be used to make the immunogens of the present disclosure.

| HIV-1 | | |
|---|---|---|
| Protein | | |
| (regions) | Subunit (amino acid residues) | Exemplary Nucleotide Sequence |
| Gag | VWASRELERFAVNPGLLETSEGCRQILGQLQ | GTATGGGCAAGCAGGGAGCTAGAACGATTCGCAGTTAATCCTGGC |
| (35-65) | (SEQ ID NO: 1) | CTGTTAGAAACATCAGAAGGCTGTAGACAAATACTGGGACAGCTACAA |
| | | (SEQ ID NO: 15) |
| Gag | QAISPRTLNAWVKVVEEKAFSPEVIPMFSALS | CAGGCCATATCACCTAGAACTTTAAATGCATGGGTAAAAGTAGT |
| (145-356) | EGATPQDLNTMLNTVGGHQAAMQMLKETINE | AGAAGAGAAGGCTTTCAGCCCAGAAGTGATACCCATGTTTTCAGC |
| | EAAEWDRLHPVHAGPIAPGQMREPRGSDIAG | ATTATCAGAAGGAGCCACCCCACAAGATTTAAACACCATGCTAAAC |
| | TTSTLQEQIGWMTNNPPIPVGEIYKRWII | ACAGTGGGGGGACATCAAGCAGCCATGCAAATGTTAAAAGAGACC |
| | LGLNKIVRMYSPTSILDIRQGPKEPFRDYV | ATCAATGAGGAAGCTGCAGAATGGGATAGATTGCATCCAGTGCATG |
| | DRFYKTLRAEQASQEVKNWMTETLLVQNANP | CAGGGCCTATTGCACCAGGCCAGATGAGAGAACCAAGGGGAAGTG |
| | DCKTILKALGPAATLEEMMTACQGVGGP | ACATAGCAGGAACTACTAGTACCCTTCAGGAACAAATAGGATGGATG |
| | (SEQ ID NO: 2) | ACAAATAATCCACCTATCCCAGTAGGAGAAATTTATAAAAGATGGATA |
| | | ATCCTGGGATTAAATAAAATAGTAAGAATGTATAGCCCTACCAGCATT |
| | | CTGGACATAAGACAAGGACCAAAGGAACCCTTTAGAGACTATGTAGA |
| | | CCGGTTCTATAAAACTCTAAGAGCCGAGCAAGCTTCACAGGAGGTAA |
| | | AAAATTGGATGACAGAAACCTTGTTGGTCCAAAATGCGAACCCAGAT |
| | | TGTAAGACTATTTTAAAAGCATTGGGACCAGCGGCTACACTAGAAGA |
| | | AATGATGACAGCATGTCAGGGAGTAGGAGGACCC |
| | | (SEQ ID NO: 16) |
| Pol | EALLDTGADDTVLEEMNLPGRWKPKMIG | GAAGCTCTATTAGATACAGGAGCAGATGATACAGTATTAGAAGAAAT |
| (77-112) | IGIGGFKV | GAATTTGCCAGGAAGATGGAAACCAAAAATGATAGGGGGAATTGGAG |
| | (SEQ ID NO: 3) | GTTTTATCAAAGTA (SEQ ID NO: 17) |
| Pol | TPDKKHQKEPPFLWMGYELHPDKWTVQ | ACACCAGACAAAAAACATCAGAAAGAACCTCCATTCCTTTGGATGGGT |
| (371-426) | PIVLPEKDSWTVNDIQKLVGKLNWASQIY | TATGAACTCCATCCTGATAAATGGACAGTACAGCCTATAGTGCTGCCAG |
| | (SEQ ID NO: 4) | AAAAAGACAGCTGGACTGTCAATGACATACAGAAGTTAGTGGGGAAATT |
| | | GAATTGGGCAAGTCAGATTTAC (SEQ ID NO: 18) |
| Vif (1-31) | MENRWQVMIVWQVDRMRIRTWKSLVKHHMYI | ATGGAAAACAGATGGCAGGTGATGATTGTGTGGCAAGTAGACAGGATGA |
| | (SEQ ID NO: 5) | GGATTAGAACATGGAAAAGTTTAGTAAAACACCATATGTATATT |
| | | (SEQ ID NO: 19) |
| Vif | DAKLVITTYVVGLHTGERDWHLGQGVSIEWRK | GATGCTAAATTGGTAATAACAACATATTGGGGTCTGCATACAGGAGAAAG |
| (61-91) | (SEQ ID NO: 6) | AGACTGGCATTTGGGTCAGGGAGTCTCCATAGAATGGAGGAAA |
| | | (SEQ ID NO: 20) |
| Env | FLGFLGAAGSTMGAASITLTVQARQLLSGIVQQ | TTCCTTGGGTTCTTGGGAGCAGCAGGAAGCACTATGGGCGCAGCCTCAA |
| (519-579) | QNNLLRAIEAQQHLLQLTVVVGIKQLQAR | TAACGCTGACGGTACAGGCCAGACAATTATTGTCTGGTATAGTGCAGCAG |
| | (SEQ ID NO: 7) | CAGAACAATTTGCTGAGGGCTATTGAGGCGCAACAGCATCTGTTGC |
| | | AACTCACAGTCTGGGGCATCAAGCAGCTCCAGGCAAGA |
| | | (SEQ ID NO: 21) |
| Env | SLCLFSYHRLRDLLLIVTRIVELLGRRGWEA | AGCCTGTGCCTCTTCAGCTACCACCGCTTGAGAGACTTACTCTTGATT |
| (762-792) | (SEQ ID NO: 8) | GTAACGAGGATTGTGGAACTTCTGGGACGCAGGGGGTGGGAAGCC |
| | | (SEQ ID NO: 22) |
| Nef | NADCAWLEAQEEEEVGFPVRPQVPLRPMTYK | AATGCTGATTGTGCCTGGCTAGAAGCACAAGAGGAGGAGGAGGTGGG |
| (52-82) | (SEQ ID NO: 9) | TTTTCCAGTCAGACCTCAGGTACCTTTAAGACCAATGACTTACAAG |
| | | (SEQ ID NO: 23) |
| Nef | YSQKRQDILDLWVYHTQGYFPDWQNYTPGPG | TACTCCCAAAAAAGACAAGATATCCTTGATCTGTGGGTCTACCACA |
| (102-132) | (SEQ ID NO: 10) | CACAAGGCTACTTCCCTGATTGGCAGAACTACACACCAGGGCCAGGG |
| | | (SEQ ID NO: 24) |

In some embodiments, one or more of the subunits is repeated in the immunogen. Any subunit may be present in 1, 2, 3, 4, 5 or more copies. Preferably, if any subunit is present more than once (i.e., repeated), the repeated subunits are flanked by different subunits relative to each other, thereby creating different junctional epitopes at each repeated subunit.

Some immunogens lack one or more of Nef subunits (SEQ ID NOs: 9 and 10) and/or Vif subunit (SEQ ID NO:5).

In some embodiments, the peptide immunogen does not include residues from the transmembrane region of gp41 and the membrane-binding region of p17 (to avoid potential protein aggregation).

In some embodiments, the immunogens may be presented as synthetic long peptides (SLPs). As used herein, a SLP comprises at least two subunits—thus is at least 62 residues in length. Methods of making SLPs are known in the art. In some embodiments, the synthetic peptides are formulated in Freund's adjuvant (FA) or aluminum phosphate (alum) to compare their ability to induce HIV-specific immune responses in mammals.

### Immunization/Vaccination

Disclosed herein are methods for immunizing (e.g., vaccinating) a subject using the peptide immunogens and/or nucleic acids encoding such peptide immunogens. These methods may be used to stimulate (or induce) an immune response in a subject. Such immune response is specific for HIV-1. Suitable subjects are those having an HIV-1 infection and those at risk of developing an HIV-1 infection.

Vaccination is a form of immunization that entails the deliberate introduction of an antigen (or immunogen, as in the case of this disclosure), in the form of a vaccine, into the body to stimulate an immune response against the administered antigen and its naturally occurring counterpart (e.g., a virus, a bacterium, etc.). These compositions may comprise microorganisms (inactivated or attenuated), or components of microorganism such as proteins, peptides, or toxins from the organism. In the present case, these compositions comprise peptide immunogens that comprise non-contiguous amino acid sequence from the HIV-1 proteome, concatenated together to form a single peptide that is itself not naturally occurring but which is nevertheless able to induce immune responses to its subunits and more importantly to HIV-1 itself.

The immune response that is induced upon administration of the immunogen may involve induction of T cells and/or B cells, including memory T cells and/or memory B cells. These immune responses are useful in reducing pathogen load in a subject, where the immunogen is directed against a pathogen, such as in the present case. Pathogen load may be reduced to the extent that pathogens are no longer detectable in the subject or in samples obtained from the subject. These immune responses may reduce symptoms associated with pathogen load. These immune responses may reduce the duration of an infection and/or may reduce the severity of the infection. When used prophylactically, the immunogen compositions may prevent a subject from developing an infection when the subject is exposed to the pathogen.

The immunogen containing compositions, whether peptide or nucleic acid in nature may be administered as a single dose or in multiple doses (e.g., a prime dose and one or more boost doses). A prime dose, sometimes referred to as primary dose or primary immunization, refers to the first administered dose of the immunogen.

A boost (or booster) dose is a second or subsequent administration of the immunogen(s). In some cases, boost doses are administered more than once. In some cases, boost doses are administered regularly (e.g., daily, weekly, monthly, every 2, 3, 4, 5, 6, 7, 8, 9, 10, 11, 12 months, yearly, every 1, 2, 3, 4, 5, 6, 7, 8, 9, 10 years, etc.).

### HIV Proteome

Human Immunodeficiency Virus (HIV) is the etiological agent of acquired human immune deficiency syndrome (AIDS) and related disorders. There are two main types of HIV: HIV-1 and HIV-2. The similarities between HIV-1 and HIV-2 include their basic gene arrangement, modes of transmission, intracellular replication pathways and clinical consequences: both result in AIDS. However, HIV-2 is known to have lower transmissibility and reduced likelihood of progression to AIDS.

The sequence diversity of HIV-1 proteins is a combination of the frequency of mutations, (e.g. about 1.4×10⁻⁵ per base pair; Abram et al., 2010), two to three recombination events per cycle of virus replication (Jetzt et al., 2000), and a high replication rate (e.g. about 10¹⁰ to 10¹² virions per day; Perelson et al., Science, 1996). This leads to the rapid evolution of genetically distinct mutant viruses, which accumulate within the host. Survival of the individual variant viruses is determined by the viral fitness and a complex association of mutations and immune escape interactions (US Publication No. 2013/0195904).

HIV-1 encodes 15 distinct proteins: the Gag and Env structural proteins MA (matrix), CA (capsid), NC (nucleocapsid), p6, SU (surface), and TM (transmembrane); the Pol enzymes PR (protease), RT (reverse transcriptase), and IN (integrase); the gene regulatory proteins Tat and Rev; and the accessory proteins Nef, Vif, Vpr, and Vpu. The HIV-1 genome encodes nine open reading frames, three of which encode the Gag, Pol, and Env polyproteins. The four Gag proteins, MA (matrix), CA (capsid), NC (nucleocapsid), and p6, and the two Env proteins, SU (surface or gp120) and TM (transmembrane or gp41), are structural components that make up the core of the virion and outer membrane envelope. The three Pol proteins, PR (protease), RT (reverse transcriptase), and IN (integrase), provide essential enzymatic functions and are also encapsulated within the particle (Frankel and Young, Annual Review of Biochemistry, 1998).

The peptide immunogens of this disclosure and their encoding nucleic acids comprise subunits from one or more of the HIV-1 Gag, Pol, Vif, and Env proteins, and optionally also from the Nef protein. In some embodiments, a peptide immunogen (or its encoding nucleic acid) comprises subunits that are selected from 2 or more of these distinct HIV proteins. In some embodiments, the peptide or nucleic acid comprises subunits from any two of the group consisting of Gag, Pol, Vif, Env, and Nef. The sequences for these proteins are known in the art.

### Nucleic Acid

The nucleic acids of the present disclosure may be provided as DNA or RNA and may comprise nucleotide sequence that encodes any of the contemplated immunogens with or without other regulatory regions such as but not limited to promoters, enhancers, etc. In some instances, the nucleic acids are nucleic acid vectors useful for delivery and/or expression of the encoded immunogen in host cells such as human cells. Examples of such vectors include such DNA vectors, RNA vectors, viral vectors, bacterial vectors, etc.

As used herein, the term “nucleic acid” refers to at least two nucleotides covalently linked together, and in some instances, may contain phosphodiester bonds (e.g., a phosphodiester “backbone”). A nucleic acid of the present disclosure may be referred to as an “engineered nucleic acid” (also referred to as a “construct”) to indicate that it does not occur in nature. It should be understood, however, that while an engineered nucleic acid as a whole is not naturally-occurring, it may include nucleotide sequences that occur in nature. In some embodiments, an engineered nucleic acid comprises nucleotide sequences from different organisms (e.g., from different species). For example, in some embodiments, an engineered nucleic acid includes an adenoviral nucleotide sequence and a retroviral (e.g., HIV-1) nucleotide sequence. Engineered nucleic acids may be recombinant nucleic acids and synthetic nucleic acids. A “recombinant nucleic acid” is a molecule that is constructed by joining nucleic acids (e.g., isolated nucleic acids, synthetic nucleic acids or a combination thereof) and, in some embodiments, can replicate in a living cell. A “synthetic nucleic acid” is a molecule that is amplified or chemically, or by other means, synthesized. A synthetic nucleic acid includes those that are chemically modified, or otherwise modified, but can base pair with naturally-occurring nucleic acid molecules. Recombinant and synthetic nucleic acids also include those molecules that result from the replication of either of the foregoing.

In some embodiments, a nucleic acid of the present disclosure is considered to be a nucleic acid analog, which may contain, at least in part, other backbones comprising, for example, phosphoramide, phosphorothioate, phosphorodithioate, O-methylphophoroamidite linkages and/or peptide nucleic acids. A nucleic acid may be single-stranded (ss) or double-stranded (ds), as specified, or may contain portions of both single-stranded and double-stranded sequence. In some embodiments, a nucleic acid may contain portions of triple-stranded sequence. A nucleic acid may be DNA, both genomic and/or cDNA, RNA or a hybrid, where the nucleic acid contains any combination of deoxyribonucleotides and ribonucleotides (e.g., artificial or natural), and any combination of bases, including uracil, adenine, thymine, cytosine, guanine, inosine, xanthine, hypoxanthine, isocytosine and isoguanine.

Nucleic acids of the present disclosure may include one or more genetic elements. A “genetic element” refers to a particular nucleotide sequence that has a role in nucleic acid expression (e.g., promoter, enhancer, terminator) or encodes a discrete product of an engineered nucleic acid (e.g., a nucleotide sequence encoding a protein).

Nucleic acids of the present disclosure may be produced using standard molecular biology methods (see, e.g., Green and Sambrook, Molecular Cloning, A Laboratory Manual, 2012, Cold Spring Harbor Press).

### Vectors

In some embodiments, an engineered nucleic acid is administered to a subject in the form of a vector. As used herein, the term “vector” refers to a nucleic acid (e.g., DNA) used as a vehicle to artificially carry genetic material (e.g., an engineered nucleic acid) into a cell where, for example, it can be replicated and/or expressed.

In some embodiments of the present disclosure, the total length of the nucleotide sequence that encodes the immunogens of the present invention is optimized for efficient expression in a vector. In such cases, the total length of the nucleotide sequence that encodes the immunogens of the present invention is typically between 300-1600 residues in length.

Any nucleic acid vector may be used including, but not limited to, plasmid vectors, retroviral vectors, lentiviral vectors, adenovirus vectors, poxvirus vectors, herpesvirus vectors and adeno-associated virus (AAV) vectors, etc. Such vectors are known in the art. See for example U.S. Pat. Nos. 6,534,261; 6,607,882; 6,824,978; 6,933,113; 6,979,539; 7,013,219; and 7,163,824, incorporated by reference herein in their entireties. When used in accordance with this disclosure, any of these vectors may comprise one or more of the multiunit immunogen nucleotide sequences provided herein. Thus, when one or more multiunit immunogens are introduced into a subject and thus into cells of the subject, the multiunit immunogens may be carried on the same vector or on different vectors. When multiple vectors are used, each vector may comprise a sequence encoding one or multiple multiunit immunogens. Similarly, when prime and boost doses are used, in some instances, the multiunit immunogen(s) presented in the prime dose may be different from the multiunit immunogen(s) presented in the boost dose (e.g., they may have a different order of subunits and/or they may have a different subset of subunits).

Conventional viral and non-viral based gene transfer methods can be used to introduce nucleic acids encoding the multiunit immunogens in cells (e.g., mammalian cells) and target tissues.

Non-viral vector delivery systems include DNA plasmids, naked nucleic acid, and nucleic acid complexed with a delivery vehicle such as a liposome or poloxamer. Viral vector delivery systems include DNA and RNA viruses, which have either episomal or integrated genomes after delivery to the cell. See for example Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds.) (1995); and Yu et al., Gene Therapy 1:13-26 (1994).

Methods of non-viral delivery of nucleic acids include electroporation, sonoporation, lipofection, microinjection, biolistics, virosomes, liposomes, immunoliposomes, polycation or lipid:nucleic acid conjugates including targeted liposomes such as immunolipid complexes, naked DNA, artificial virions, and agent-enhanced uptake of DNA. See for example U.S. Pat. Nos. 4,186,183; 4,217,344; 4,235,871; 4,261,975; 4,485,054; 4,501,728; 4,774,085; 4,837,028; 4,946,787; 6,008,336; 5,049,386; 4,946,787; and 4,897,35; and published PCT applications WO 91/17424; and WO 91/16024.

This disclosure contemplates integration of the immunogen encoding nucleic acid sequences into the genome of a host cell, thereby providing long-term expression, as well as non-integration of such sequences, thereby providing more transient expression. The immunogens may be expressed for days (e.g., 1-31 days or any number of days or ranges of days in between), weeks (e.g., 1-4 weeks, or any number of weeks or ranges of weeks in between), months (e.g., 1-12 months or any number of months or ranges of months in between), or years (e.g., 1 year, 2 years, 3 years, 4 years, 5 years, etc.).

In applications in which transient expression is preferred, adenoviral based systems can be used. Adenoviral based vectors are capable of very high transduction efficiency and do not require cell division. With such vectors, high titer and high levels of expression have been obtained. This vector can be produced in large quantities in a relatively simple system. Adeno-associated virus (“AAV”) vectors are also used to transduce cells with target nucleic acids, for in vitro use, in vivo use and/or ex vivo use (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Recombinant adeno-associated virus vectors (rAAV) are based on the defective and nonpathogenic parvovirus adeno-associated type 2 virus. All vectors are derived from a plasmid that retains only the AAV 145 bp inverted terminal repeats flanking the transgene expression cassette. Other AAV serotypes, including AAV1, AAV3, AAV4, AAVS, AAV6, AAV8, AAV 8.2, AAV9, AAV rh10 and pseudotyped AAV such as AAV2/8, AAV2/5 and AAV2/6 can also be used in accordance with the present disclosure.

Replication-deficient recombinant adenoviral vectors (Ad) can be produced at high titer and readily infect a number of different cell types. Most adenovirus vectors are engineered such that a transgene replaces the Ad E1a, E1b, and/or E3 genes. The replication defective vector is propagated in human cells (e.g., 293 cells) that supply deleted gene function in trans. Ad vectors can transduce multiple types of tissues in vivo, including non-dividing, differentiated cells such as those found in liver, kidney and muscle. Conventional Ad vectors have a large carrying capacity.

A non-limiting example of a vector is a plasmid, which is a double-stranded, generally circular, DNA sequence that is capable of automatically replicating in a host cell. Plasmid vectors typically contain an origin of replication that allows for semi-independent replication of the plasmid in the host and also the transgene insert. Plasmids may have more features, including, for example, a “multiple cloning site,” which includes nucleotide overhangs for insertion of a nucleic acid insert, and multiple restriction enzyme consensus sites to either side of the insert. In some embodiments, the vector is a DNA or RNA vector.

Another non-limiting example of a vector is a viral vector. Thus, in some embodiments, the nucleic acid of the present disclosure is delivered to the cells of a subject using a viral delivery system (e.g., retroviral, adenoviral, adeno-association, helper-dependent adenoviral systems, hybrid adenoviral systems, herpes simplex, pox virus, lentivirus, Epstein-Barr virus) or a non-viral delivery system (e.g., physical: naked DNA, DNA bombardment, electroporation, hydrodynamic, ultrasound or magnetofection; or chemical: cationic lipids, different cationic polymers or lipid polymer) (Nayerossadat N et al. Adv Biomed Res. 2012; 1: 27, incorporated herein by reference). In some embodiments, the non-viral based deliver system is a hydrogel-based delivery system (see, e.g., Brandl F, et al. Journal of Controlled Release, 2010, 142(2): 221-228, incorporated herein by reference).

Nucleic acid vectors can be delivered in vivo by administration to a subject (e.g., human patient), typically by systemic administration (e.g., intravenous, intraperitoneal, intramuscular, subdermal, or intracranial infusion) or topical application, as described below. Alternatively, vectors can be delivered to cells ex vivo, such as cells explanted from a subject, followed by re-implantation of the cells into a subject, optionally after selection for cells which have incorporated the vector.

Vectors (e.g., retroviruses, adenoviruses, liposomes, etc.) encoding the multiunit immunogens can also be administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation.

### Adenoviral or Adeno-Associated Viral Vectors

In some embodiments, the nucleic acid of the present disclosure is an adenoviral vector or an adenovirus-associated viral vector. In preferred embodiments, the adenoviral vector of the present disclosure is a replication incompetent adenoviral vectors. In alternative embodiments, the adenoviral vector of the present disclosure is a replication competent adenoviral vector. The adenovirus genome is a linear double stranded DNA. It comprises early-transcribed regions E1, E2, E3, and E4. The E1 region (which includes E1A and E1B) encodes proteins that are involved in replication. Thus, a replication incompetent adenoviral vector can be made by deleting the E1 region. In many replication incompetent adenoviral vectors, the E1 region is deleted and replaced with an expression cassette with an exogenous promoter that drives expression of the exogenous therapeutic gene. Modification of an adenoviral vector to yield replication incompetence allows for safe gene delivery.

Adenoviral vectors can be used to produce high titers (e.g. 10E10 VP/mL, 10E13 VP/mL) and can incorporate large transgenes (e.g. up to 8 kb). They are capable of infecting most mammalian cells and are not integrated into the host chromosome. The major disadvantage of adenoviral vectors is that they can be highly immunogenic, eliciting an immune response against the vector genome (antivector immunity). The use of rare serotypes can help minimize the risks associated with antivector immunity. Additionally, the use of different serotype viral vectors in the prime and boost doses of the present disclosure minimizes the risk associated with antivector immunity.

In some embodiments, the nucleic acid vectors of the present disclosure are adenoviral vectors derived from a human serotype. As used herein, a “serotype” (also referred to as serovar) refers to a distinct variation within a species of bacteria or virus or among immune cells of different individuals. There are at least 57 serotypes of human adenovirus (Ads), e.g. Ad1-Ad57, that form seven “species” A-G. In some embodiments, an adenoviral vector from any one the seven species A-G is used. The most common human Ads serotypes are from Species C (e.g. Ad1, Ad2, Ad5, and Ad6). Rare human Ads serotypes that are contemplated herein include, but are not limited to, Ad26, Ad48, and Ad49. Non-limiting examples of adenoviral serotypes include Ad5, Ad11, Ad35, Ad50, Ad26, Ad48, and Ad49 (see, for example, Abbink et al. Journal of Virology, 2007).

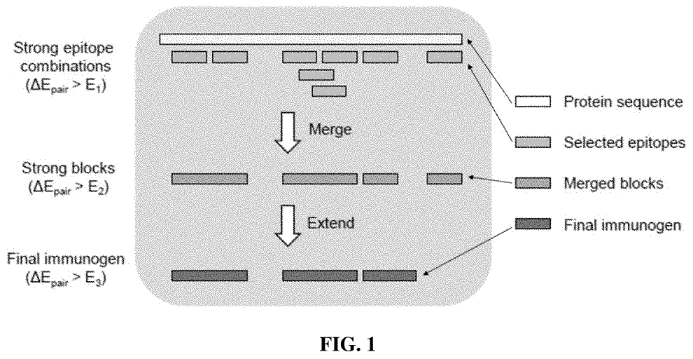

In some embodiments, the nucleic acid vectors of the present disclosure are derived from rhesus adenovirus. Non-limiting examples of rhesus-derived adenovirus serotypes include RhAd51, RhAd52 or RhAd53. Additional examples of rhesus-derived adenovirus serotypes are provided in Abbink et al. Journal of Virology, 2018 (FIG. 1 and Table 1).

In some embodiments, the adenoviral vector serotype is a serotype having lower seroprevalence in the human population or in the subject relative to a human serotype adenoviral vector. The seroprevalence in the human population can be determined based on region (e.g. sub-Saharan populations, western populations, etc.)

### Compositions

The immunogens of this disclosure, whether in peptide or nucleic acid form, may be provided in compositions together with one or more other components. Such compositions may be used in vitro, in vivo or ex vivo.

In some embodiments, the immunogens or nucleic acids of the present disclosure may be formulated in a composition for administering to a subject. In some embodiments, the composition is a pharmaceutical composition. In some embodiments, the composition further comprises additional agents (e.g. for specific delivery, increasing half-life, or other therapeutic agents). In some embodiments, the composition further comprises a pharmaceutically acceptable carrier. The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. A “pharmaceutically acceptable carrier” is a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation.

Some examples of materials which can serve as pharmaceutically-acceptable carriers include, without limitation: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as peptides and amino acids (23) serum component, such as serum albumin, HDL and LDL; (24) C2-C12 alcohols, such as ethanol; and (25) other non-toxic compatible substances employed in pharmaceutical formulations. Wetting agents, coloring agents, release agents, coating agents, sweetening agents, flavoring agents, perfuming agents, preservative and antioxidants can also be present in the formulation.

Compositions (e.g. vaccines) containing peptides are generally well known in the art, as exemplified by U.S. Pat. Nos. 4,601,903; 4,599,231; 4,599,230; and 4,596,792. In some embodiments, the compositions are prepared as injectables, as liquid solutions or emulsions. The peptides may be mixed with pharmaceutically-acceptable excipients which are compatible with the peptides. Excipients may include water, saline, dextrose, glycerol, ethanol, and combinations thereof. The compositions may further contain auxiliary substances such as wetting or emulsifying agents, pH buffering agents, or adjuvants to enhance the effectiveness of the vaccines. Methods of achieving adjuvant effect for the compositions (e.g. vaccines) include the use of adjuvants such as aluminum hydroxide or phosphate (alum), commonly used as 0.05 to 0.1 percent solution in phosphate buffered saline.

### Treatment

Disclosed herein are methods for treating a subject having an HIV-1 infection, referred to as therapeutic treatment of the subject. In some embodiments, the subject has acquired immunodeficiency syndrome (AIDS). The disclosed methods for treating a subject having an HIV-1 infection comprise administering to said subject an effective amount (e.g. a therapeutically effective amount) of a peptide immunogen of the present disclosure (or its encoding nucleic acid). A effective amount is a dose sufficient to provide a medically desirable result and can be determined by one of skill in the art using routine methods, and in discussed in greater detail below. The art is familiar with identification and thus diagnosis of subjects having an HIV-1 infection.

Also disclosed herein are methods for treating a subject at risk of having an HIV-1 infection, also referred to as a prophylactic treatment. The methods comprise administering to said subject an effective amount of a peptide immunogen of the present disclosure (or its encoding nucleic acid). Subjects at risk of having (or developing an HIV-1 infection include those exposed to HIV-1-positive individuals, those receiving transfusion or transplants including transfusions or transplants from subjects who are HIV-1 positive, those born to HIV-1-positive mothers, those engaging in high risk activity such as intravenous drug use, etc.

These treatment methods may comprise administering the peptide immunogens or nucleic acids in a prime dose and a boost dose. As used herein, “a prime dose” refers to an initial administration of a peptide or nucleic acid of the present disclosure to a subject. As used herein, a “boost dose” refers to one or more subsequent administrations of a peptide or nucleic acid of the present disclosure. In some embodiments, the prime dose and boost dose have different immunogens (e.g., different shuffled versions, different orders of subunits, different subsets of subunits, etc.). Preferably, these different immunogens have different junctional epitopes. For example, the subunits within the immunogen in the boost dose have different subunit order (i.e., are shuffled) relative to the prime dose in such a way that there is no recurrence of a junctional epitope, as described herein. (In other words, each junctional epitope is present only once over all of the immunogens that are ultimately administered to a subject.) In some cases the boost immunogen has no recurring junctional epitopes (i.e. relative to the prime dose or a previous dose).

The use of different (e.g., shuffled) versions of immunogens in a prime-dose treatment regimen reduces the likelihood of inducing immune responses against the non-naturally junctional epitopes.

In some embodiments, the use of different serotype viral vectors in the prime and boost doses of the present disclosure minimizes the risk associated with antivector immunity (e.g. ineffective treatment). Alternatively, an adenoviral vector can be used for either the prime or boost dose and a different type of vector can be used for the other dose. In some embodiments, an adenoviral vector is used for either the prime or boost dose, and a peptide immunogen is used for the other dose. These minimize the risks associated with antivector immunity and can yield a more potent (effective) immune response. Potency of the immune response can be measure used methods in the art to measure immune response.

### Secondary Therapies/Second Therapeutic Agents

In some embodiments, subjects may be administered an anti-retroviral agent. An anti-retroviral agent is an agent that specifically inhibits a retrovirus from replicating or infecting cells. Non-limiting examples of antiretroviral drugs include entry inhibitors (e.g., enfuvirtide), CCR5 receptor antagonists (e.g., aplaviroc, vicriviroc, maraviroc), reverse transcriptase inhibitors (e.g., lamivudine, zidovudine, abacavir, tenofovir, emtricitabine, efavirenz), protease inhibitors (e.g., lopivar, ritonavir, raltegravir, darunavir, atazanavir), maturation inhibitors (e.g., alpha interferon, bevirimat and vivecon).

In some instances, the subject may be administered at least one anti-retroviral agent (e.g., one, two, three or four anti-retroviral agents). One example of a combination of anti-retroviral agents is a combination of tenofovir, emtricitabine and efavirenz.

Other classes of antiretroviral drugs include nucleoside analog reverse-transcriptase inhibitors (such as zidovudine, didanosine, zalcitabine, stavudine, lamivudine, abacavir, emtricitabine, entecavir, and apricitabine), nucleotide reverse transcriptase inhibitors (such as tenofovir and adefovir), non-nucleoside reverse transcriptase inhibitors (such as efavirenz, nevirapine, delavirdine, etravirine, and rilpivirine), protease inhibitors (such as saquinavir, ritonavir, indinavir, nelfinavir, amprenavir, lopinavir, fosamprenavir, atazanavir, tipranavir, and darunavir), entry or fusion inhibitors (such as maraviroc and enfuvirtide), maturation inhibitors, (such as bevirimat and vivecon), or a broad spectrum inhibitors, such as natural antivirals. Any one or any combination of the foregoing agents may be used in accordance with this disclosure.

### Adjuvants

In some embodiments, the immunogens of this disclosure may be administered with one or more adjuvants. The adjuvant may be without limitation alum (e.g., aluminum hydroxide, aluminum phosphate); saponins purified from the bark of the Q. saponaria tree such as QS21 (a glycolipid that elutes in the 21st peak with HPLC fractionation; Antigenics, Inc., Worcester, Mass.); poly[di(carboxylatophenoxy)phosphazene (PCPP polymer; Virus Research Institute, USA), Flt3 ligand, Leishmania elongation factor (a purified Leishmania protein; Corixa Corporation, Seattle, Wash.), ISCOMS (immunostimulating complexes which contain mixed saponins, lipids and form virus-sized particles with pores that can hold antigen; CSL, Melbourne, Australia), Pam3Cys, SB-AS4 (SmithKline Beecham adjuvant system #4 which contains alum and MPL; SBB, Belgium), non-ionic block copolymers that form micelles such as CRL 1005 (these contain a linear chain of hydrophobic polyoxypropylene flanked by chains of polyoxyethylene, Vaxcel, Inc., Norcross, Ga.), and Montanide IMS (e.g., IMS 1312, water-based nanoparticles combined with a soluble immunostimulant, Seppic)

Adjuvants may be TLR ligands. Adjuvants that act through TLR3 include without limitation double-stranded RNA. Adjuvants that act through TLR4 include without limitation derivatives of lipopolysaccharides such as monophosphoryl lipid A (MPLA; Ribi ImmunoChem Research, Inc., Hamilton, Mont.) and muramyl dipeptide (MDP; Ribi) andthreonyl-muramyl dipeptide (t-MDP; Ribi); OM-174 (a glucosamine disaccharide related to lipid A; O M Pharma S A, Meyrin, Switzerland). Adjuvants that act through TLRS include without limitation flagellin. Adjuvants that act through TLR7 and/or TLR8 include single-stranded RNA, oligoribonucleotides (ORN), synthetic low molecular weight compounds such as imidazoquinolinamines (e.g., imiquimod (R-837), resiquimod (R-848)). Adjuvants acting through TLR9 include DNA of viral or bacterial origin, or synthetic oligodeoxynucleotides (ODN), such as CpG ODN. Another adjuvant class is phosphorothioate containing molecules such as phosphorothioate nucleotide analogs and nucleic acids containing phosphorothioate backbone linkages.

### Modes of Administration

The peptide immunogens and nucleic acid constructs of the present disclosure may be administered to a subject in need of the treatment via a suitable route (e.g., intramuscular injection or local injection). Similarly, any of the peptide immunogens and nucleic acid constructs of the present disclosure can be delivered to a subject in need of the treatment via a suitable route. In some embodiments, the peptide immunogens and nucleic acid constructs of the present disclosure can be administered parentally, intravenously, intradermally, intraarterially, intralesionally, intratumorally, intracranially, intraarticularly, intraprostaticaly, intrapleurally, intratracheally, intranasally, intravitreally, intravaginally, intrarectally, topically, intramuscularly, by puncture, intraperitoneally, subcutaneously, subconjunctival, intravesicularlly, mucosally, intrapericardially, intraumbilically, intraocularally, orally, locally, inhalation (e.g., aerosol inhalation), transdermally, by injection, infusion, continuous infusion, localized perfusion bathing target cells directly, via a catheter, via a lavage, in creams, in lipid compositions (e.g., liposomes), or by other method or any combination of the forgoing as would be known to one of ordinary skill in the art (see, for example, Remington's Pharmaceutical Sciences (1990), incorporated herein by reference).

### Effective Amount

The compositions of the present disclosure are administered in a manner compatible with the dosage formulation. In some embodiments, a subject having or at risk of having an HIV-1 infection is administered an effective amount of a peptide immunogen of the present disclosure. In alternative embodiments, a subject having or at risk of having an HIV-1 infection is administered an effective amount of a nucleic acid of the present disclosure. As used herein, the term “effective amount” refers to an amount sufficient to stimulate an immune response to the antigen in the subject. In some embodiments, said immune response is a CD8⁺ T-lymphocyte response specific for one or more targeted epitopes in the immunogen. In some embodiments, said immune response is an increase in antibodies specific for the targeted epitopes. In some embodiments, the effective amount may decrease the subject's viral load, including reducing to undetectable levels. The immunogen may be administered in an amount sufficient to alleviate the symptoms of HIV or a secondary infection or condition such as for example AIDS.

When administered to a subject, effective amounts of the immunogen, whether administered as a peptide or a nucleic acid, will depend, of course, on the severity of the disease (e.g. the current viral load of the subject); individual patient parameters including age, physical condition, size and weight, concurrent treatment, frequency of treatment, and the mode of administration. These factors are well known to those of ordinary skill in the art and can be addressed with no more than routine experimentation. In some embodiments, a maximum dose is used, that is, the highest safe dose according to sound medical judgment.

Methods for detecting/diagnosing HIV infection are known in the art. Non-limiting examples of methods for detecting HIV infection include antibody tests, antigen/antibody tests, and nucleic acid tests (NATs).

An immune response may be measured by any methods known in the art, e.g., by measuring the antibody titers against the epitopes in the immunogen, measuring cytokine production or T cell activation in the subject upon administering the immunogen of the present disclosure either in its peptide form or its encoding nucleic acid form. Non-limiting examples of methods for measuring the immune response to the immunogen of the present disclosure include pooled peptide IFN-γ enzyme-linked immunospot assays (ELISPOT) assays and ELISAs at multiple time points following immunization (for example, see U.S. Application No. 6,787,351 and Abbink et al. Journal of Virology, 2007).

### (i) The ELISPOT Assay

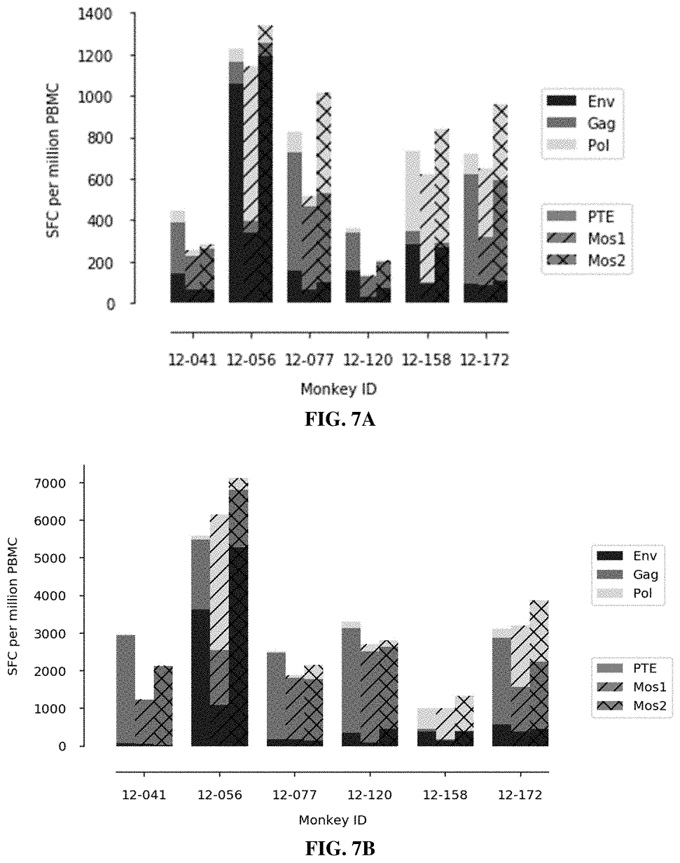

The ELISPOT assay is a quantitative determination of IV-specific T lymphocyte responses by visualization of gamma interferon secreting cells in tissue culture microtiter plates a period (e.g. one day) following addition of the peptide immunogen pool that to peripheral blood mononuclear cell (PBMC) samples. The number of spot forming cells (SPC) per million of PBMCs is determined for samples in the presence and absence (media control) of peptide antigens. The assay may be set up to determine overall T lymphocyte responses (both CD8+ and CD4+) or for specific cell populations by prior depletion of either CD8+ or CD4+ cells. In addition, the assay can be varied so as to determine which peptide epitopes are recognized by particular individuals. The experimental data provided in FIGS. 7A and 7B used three different peptide pools denoted PTE, Mos 1 and Mos2, which were shown as the first, second and third bars of each triplet.

### (ii) Cytotoxic T Lymphocyte Assays

In this assay, PBMC samples are infected with recombinant vaccinia viruses expressing gag antigen in vitro for approximately 14 days to provide antigen restimulation and expansion of memory T cells. The cells are then tested for cytotoxicity against autologous B cell lines treated with peptide antigen pools. The phenotype of responding T lymphocytes is determined by appropriate depletion of either CD8+ or CD4+ cells.

The quantity to be administered depends on the subject to be treated, including, for example, the capacity of the individual's immune system to synthesize antibodies, and to produce a cell-mediated immune response. The effective amount of active ingredient required to be administered depends on the judgment of the practitioner. However, suitable dosage ranges are readily determinable by one skilled in the art—in some embodiments, they are of the order of micrograms of the peptides. Suitable regimes for initial administration and booster doses are also variable, but may include an initial administration followed by subsequent administrations, for example, at least one pre-peptide immunization with a non-infectious, non-replicating viral vector, followed by at least one secondary immunization with the peptides provided herein. The dosage of the vaccine may also depend on the route of administration and will vary according to the size of the host.

### Subject

In some embodiments of the present disclosure, the term “subject” refers to a mammal. In some embodiments the subject is a human or human patient. In some embodiments, the subject is an animal (e.g., animal model). In other embodiments the subject is a mouse. In other embodiments, the subject is a monkey (e.g. rhesus monkey). Subjects also include animals such as household pets e.g., dogs, cats, rabbits, ferrets, etc.), livestock or farm animals (e.g., cows, pigs, sheep, chickens and other poultry), horses such as thoroughbred horses, laboratory animals (e.g., rats, rabbits, etc.), and the like.

The subjects to whom the agents are delivered may be normal (uninfected) subjects (e.g. patients not infected with HIV-1). The subjects may be at risk of contracting HIV-1. In some embodiments, the subject is an infant or pediatric patient. In alternative embodiments, the subject is an adult.

Subjects having an infection are those that exhibit symptoms thereof including without limitation fever, chills, myalgia, photophobia, fatigue, sore throat, pharyngitis, night sweats, acute lymphadenopathy, splenomegaly, mouth ulcers, gastrointestinal upset, leukocytosis or leukopenia, and/or those in whom infectious pathogens (e.g. HIV-1) or byproducts thereof can be detected.

A subject at risk of developing an infection is one that is at risk of exposure to an infectious pathogen (e.g. HIV-1). Such subjects include those that live in an area where such pathogens are known to exist and where such infections are common. These subjects also include those that engage in high risk activities such as sharing of needles, engaging in unprotected sexual activity, routine contact with infected samples of subjects (e.g., medical practitioners), people who have undergone surgery (including but not limited to abdominal surgery, etc.), and people who have undergone blood transfusions or dialysis.

The subject may have an HIV-1 infection or may be at risk of developing an HIV-1 infection. In some embodiments, the compositions of the present disclosure may be administered with an adjuvant (e.g. an anti-viral agent). Such an adjuvant may be useful for stimulating an immune response against the infection, or potentially treating the infection.

### II. Computational Techniques

Aspects of the present application relate to computational techniques for designing immunogens and their associated vectors, including those discussed above. One challenge in designing immunogens is identifying particular residues in viral proteins to include as epitopes in the resulting immunogen such that the immunogen targets vulnerable regions of the viral proteins. This is particularly challenging in developing vaccines for viruses that have high mutability, such as HIV. Some conventional techniques for designing immunogens involve identifying highly conserved regions of viral proteins and including those conserved regions in the immunogen. For example, the conserved regions may be determined by analyzing samples extracted from diverse patients and determining regions of the virus' proteome that are highly conserved across the patients. However, the inventors have recognized and appreciated that these techniques fail to take into account any fitness landscape effects of coupling between mutations of a target virus, particularly in viruses that have high replication and mutation rates, such as HIV. For example, a virus may evolve to have mutations that can partially restore any fitness cost incurred by mutations occurring within a region targeted by an immunogen, allowing the overall fitness of the virus to remain substantially the same. Accordingly, some embodiments of the technology described herein are directed to techniques for designing immunogens that include epitopes where mutations are especially deleterious by taking into account coupling between mutations of the target virus. Using such techniques, regions of the viral proteome determined to be particularly deleterious mutation regions may be included in the resulting immunogen while compensatory mutation regions of the viral proteome may be limited or excluded from the immunogen.

In addition, some conventional techniques for designing immunogens involve evaluating epitopes as candidates to include in an immunogen individually without evaluating the combined characteristics of multiple epitopes. Accordingly, the inventors have developed new computational techniques for determining a combination of epitopes that takes into account fitness contributions between multiple epitopes. In particular, these computational techniques may involve computing fitness costs for multiple epitopes collectively rather than for single epitopes.

The inventors have further appreciated and recognized that highly deleterious mutation regions of a viral protein sequence can be widely interspaced and that it is desirable to select very long, contiguous regions that have a high expected fitness cost. Some embodiments involve using the combination of epitopes to generate subunits of the viral protein sequences by extending beyond the combination of epitopes to lengthen the sequence that is included in the immunogen while balancing fitness costs associated with including those additional residues. In some embodiments, generating the subunits may involve reducing the presence of junctional epitopes occurring in the immunogen. In some instances, these techniques may involve generating subunits with residue lengths that are at least a desired minimum length such that the number of target epitopes exceeds the number of junctional epitopes. In some embodiments, the generated subunits may have a length of at least 31 residues.

Herein, the fitness landscape may be used to compute the fitness cost of double mutations in pairs of non-overlapping epitopes, averaged over all sequence backgrounds, which may be referred to as the “pairwise fitness cost” (for a given pair of epitopes), and used to predict pairs of epitopes wherein simultaneous mutations would be deleterious for the virus across multiple sequence backgrounds. Thus, if targeted simultaneously by a T cell response, the virus would be cornered between being killed by the T cell response or evolving unviable mutations. The pairwise fitness cost has contributions from direct fitness effects as well as from interactions with sequence background and interactions between the two epitopes. As used herein, the term “average pairwise fitness cost” (of the immunogen) refers to the average of the “pairwise fitness cost” over all pairs of non-overlapping epitopes in the immunogen.

Fitness cost of an epitope is influenced by the sequence background. The calculation may account for epistatic interactions, specifically, the synergistic (or antagonistic) interactions between mutations.

In the case of a virus, e.g. HIV-1, the prevalence order is statistically similar to the fitness landscape. This allows the inference of the fitness landscape from prevalence data. Under this assumption, epitopes that are immunoprevalent and slow to escape have the highest fitness. Such epitopes would ideally be targeted by an immune response.

As discussed herein, some embodiments of the present application may involve designing treatments that target particular viruses. The regions of a viral proteome considered to be particularly vulnerable to mutations as determined by implementing the computational techniques described herein may be incorporated into an immunogen for the target virus. In some embodiments, the immunogen may be a single polypeptide that includes these deleterious mutation regions. Some embodiments involve designing a nucleic acid that encodes for the immunogen as a treatment for a patient.

The inventors have further appreciated and recognized that particular vectors may have constraints on the characteristics of the immunogen it encodes to allow for the immunogen to be efficiently expressed. In particular, some vectors may impose a constraint on the range of the total residue length of the immunogen to allow for efficient expression of the immunogen. For example, when the adenoviral vector is used for treatment, the total length of the construct may be between 300-1600 residues to allow for efficient expression of the construct. Accordingly, some embodiments described herein involve designing an immunogen that complies with one or more constraints imposed by the vector being used as part of the treatment.

Some embodiments described herein address all of the above-described issues that the inventors have recognized with designing immunogens. However, not every embodiment described herein addresses every one of these issues, and some embodiments may not address any of them. As such, it should be appreciated that embodiments of the technology described herein are not limited to addressing all or any of the above-discussed issues with designing immunogens. It should be appreciated that the various aspects and embodiments described herein be used individually, all together, or in any combination of two or more, as the technology described herein is not limited in this respect.

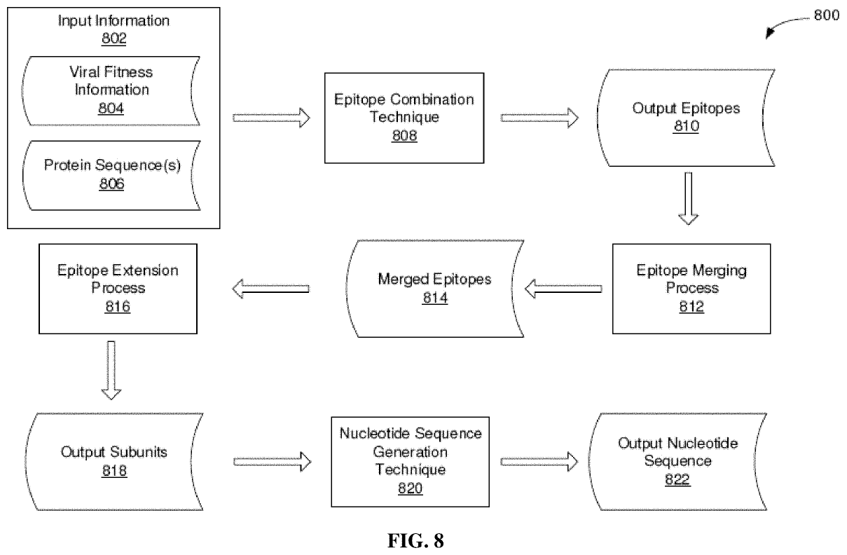

FIG. 8 is a diagram of an illustrative processing pipeline 800 for designing immunogens, which may include using viral fitness information and protein sequence(s) corresponding to protein(s) of a virus to determine a combination of epitopes as having a high fitness cost, and generating an output indicating subunits that have sequences of the epitopes, in accordance with some embodiments of the technology described herein. As shown in FIG. 8, input information 802, including viral fitness information 804 and protein sequence(s) 806, may be analyzed using epitope combination technique 808 to generate a combination of output epitopes 810.

Viral fitness information 804 may include information obtained from multiple sequences of the viral protein(s) of interest. In some instances, viral fitness information 804 may indicate a “fitness landscape” of the viral protein(s) that describes the intrinsic fitness of the viral protein(s) as a function of sequence and takes into account the effects of coupling between mutations located at different regions of the protein sequence(s) 806. Examples of fitness landscapes that may be used as viral fitness information 804 for HIV are described in Ferguson A L, et al. Translating HIV sequences into quantitative fitness landscapes predicts viral vulnerabilities for rational immunogen design, Immunity 38(3): 606-617, 21 Mar. 2013; Barton J P, et al. Relative rate and location of intra-host HIV evolution to evade cellular immunity are predictable, Nature Communications 7: 11660, 23 May 2016; and Louie R H Y, et al. Fitness landscape of the human immunodeficiency virus envelope protein that is targeted by antibodies, Proc Natl Acad Sci USA 115(4): E564-E573, 23 Jan. 2018, each of which are incorporated by reference in its entirety.

Protein sequence(s) 806 may include amino acid sequence(s) corresponding to protein(s) of a virus. In some embodiments, the virus is HIV and protein sequence(s) 806 include the set of proteins that form HIV, which are described herein. Although discussion of these computational techniques are described in the context of designing immunogens to target HIV, it should be appreciated that these techniques may be implemented in designing immunogens for other target viruses.

Epitope combination technique 808 may involve using viral fitness information 804 to determine a combination of epitopes occurring in protein sequence(s) 806 as having a high fitness cost to include as output epitopes 810. A schematic illustrating the process of determining output epitopes 810 is shown in FIG. 1. A high fitness cost may correspond to a combination of epitopes where mutations occurring within the epitopes have a deleterious effect on the virus. In some embodiments, epitope combination technique 808 may involve computing fitness cost values for different sets of epitopes occurring in protein sequence(s) by using viral fitness information 804 and evaluating which epitopes to include in the combination of output epitopes 810 based on the computed fitness cost values. To account for coupling mutations in different regions of protein sequence(s) 806, epitope combination technique 808 may involve determining contributions from direct fitness effects as well as from interactions with sequence background and interactions between two or more epitopes. In some embodiments, a fitness cost may be computed for different pairs of epitopes, which may be referred to as a “pairwise fitness cost,” and the computed fitness costs may be used in determining the combination of epitopes to include as output epitopes 810.

According to some embodiments, epitope combination technique 808 may involve performing an iterative process in computing fitness costs associated for different sets of epitopes. Some embodiments may include determining an initial set of epitopes (e.g., a pair of epitopes) as having a high fitness cost and iteratively selecting from the remaining epitopes in protein sequence(s) 806 to include in the output combination of epitopes 810. This iterative process may be repeated until the addition of another epitope to the selected combination would decrease the fitness cost to below a threshold value. At that point in the iterative process, the epitope that lowers the fitness cost below the threshold value may be excluded from the output combination of epitopes and the iterative process would output the previously considered epitopes.

In some embodiments, epitope combination technique 808 may involve determining an initial pair of epitopes as having a high fitness cost to include in output epitopes 810. The initial pair of epitopes may be determined by computing pairwise fitness cost values for pairs of non-overlapping epitopes and using the fitness cost values to determine a pair of epitopes as having a pairwise fitness cost greater than a threshold value, E₁. Epitope combination technique 808 may further involve selecting one or more additional epitopes to include as output epitopes 810 by comparing a fitness cost for a set of epitopes that includes the first pair and the one or more additional epitopes and determining which epitopes to include as output epitopes 810 based on the comparing. The fitness cost may be determined by averaging the pairwise fitness cost over all pairs of epitopes, which may be referred to as an “average pairwise fitness cost.” In some embodiments, epitope combination technique 808 may involve determining an initial pair of epitopes and one or more additional epitopes to include in output epitopes 810 if the fitness cost is above the threshold value, E₁. In some embodiments, epitope combination technique 808 may involve determining to include the initial pair of epitopes and to exclude the one or more additional epitopes in output epitopes 810 if the fitness cost is below the threshold value, E₁. In some embodiments, the value for E₁ is 8.5.

Additional discussion for how the pairwise fitness cost is calculated is described further below with respect to equations (1) and (2).

For this discussion, let s denote a sequence, and E(s) the corresponding energy. The value of the energy correlates negatively with the fitness of the viral strain with sequence s [1,2,3]. The full sequence s can be divided into two parts, sₑ, the region containing the epitope of interest, and sᵣ, which contains the rest of the protein, and the epitope sequence itself can be called e.

To average over the possible sequence backgrounds sᵣ in which the epitope e might appear, the energy/fitness cost of physically realizable mutations at different points in the epitope given all possible sequence backgrounds (e.g., sampled by a Monte Carlo procedure) may be computed, and the average fitness cost for evolving mutations at the epitope under consideration may be computed. First, the region containing the epitope may be fixed to be equal to the that of the targeted epitope, sₑ=e. The average energy difference δE(s′ₑ, sₑ) between a mutant s′ₑ and the unmutated epitope sₑ=e is

$$δ E ( {\underline{s}}_{e}^{′} , {\underline{s}}_{e} ) = 〈 E ( \{ {\underline{s}}_{r} , {\underline{s}}_{e}^{′} \} ) - E ( \{ {\underline{s}}_{r} , {\underline{s}}_{e} \} ) 〉 = {\sum }_{{\underline{s}}_{r}} [ E ( \{ {\underline{s}}_{r} , {\underline{s}}_{e}^{′} \} ) - E ( \{ {\underline{s}}_{r} , {\underline{s}}_{e} \} ) ] {e}^{- E ( \{ {\underline{s}}_{r} , {\underline{s}}_{e} \} )} . \qquad ( 1 )$$

The form of δE(s′ₑ, sₑ) may allow for estimation using suitable estimation techniques, such as via Monte Carlo. Contributions to the energy from fields and couplings between sites in sᵣ cancel, and the contribution from fields and couplings between sites entirely in sₑ is constant. The contribution to the energy from couplings between sites in sₑ and sᵣ may be computed which requires the one-point correlations for sites in sᵣ when sₑ=e is held fixed.

The estimated fitness cost of evolving escape mutations in the epitope is

$$〈 Δ {E}^{′} 〉 = {\sum }_{{\underline{s}}_{e}^{′}} δ E ( {\underline{s}}_{e}^{′} , {\underline{s}}_{e} ) w ( {\underline{s}}_{e}^{′} ) \text{/} {\sum }_{{\underline{s}}_{e}^{′}} w ( {\underline{s}}_{e}^{′} ) , where w ( {\underline{s}}_{e}^{′} ) = {e}^{- δ E ( {\underline{s}}_{e}^{′} , {\underline{s}}_{e} )} . \qquad ( 2 )$$

This average may be used for computing the average fitness cost of mutations in order to put the most weight on low energy escape routes.

Returning to FIG. 8, output epitopes 810 may include a combination of epitopes that includes epitopes accounting for coupling mutations of protein sequence(s) 806. In some embodiments, output epitopes 810 may include a combination of epitopes that includes one or more deleterious mutation regions of protein sequence(s) 806. In the context of HIV, output epitopes 810 may include one or more of the epitopes discussed herein.

Some embodiments may involve determining output subunits 818 of protein sequence(s) 806 that include output epitopes 810. As shown in FIG. 8, output epitopes 810 may be further processed by using epitope merging process 812 and epitope extension process 816 to generate output subunits 818. Output epitopes 810 may each have a residue length below a desired length. For example, some embodiments involve determining output epitopes 810 having eleven residues. In generating output subunits 818 to include in the immunogen, it may be desirable to extend the length of the protein sequence regions to include in the subunits. According to some embodiments, epitope merging process 812 may involve identifying multiple epitopes as being overlapping and merging those epitopes as being a single subunit. For example, epitopes having a residue length of 11, epitopes that overlap by 10 or less residues may be considered as overlapping and merged by epitope merging process 812.

According to some embodiments, epitope merging process 812 may involve bridging multiple non-contiguous epitopes by considering intervening amino acids between successive epitopes. A schematic illustrating the process of determining merged epitopes 812 is shown in FIG. 1. In some embodiments, epitope merging process 812 may involve determining one or more residues of protein sequence(s) 806 to include in output subunits 818 that exist outside the combination of output epitopes 810. In evaluating the intervening amino acids, the fitness cost associated with including those additional amino acids in the resulting immunogen may be considered. In some embodiments, a fitness cost associated with including one or more residues located between successive epitopes in output subunits 818 may be computed and compared to a threshold value, E₂. If the computed fitness cost is below the threshold value, then the one or more residues may be included in the output subunits 818. If the fitness cost exceeds the threshold value, then the one or more residues may be excluded from output subunits 818. Epitope merging process 812 may perform evaluation of additional residues to include in output subunits 818 through an iterative process to arrive at a set of output subunits that has a fitness cost that meets the threshold value, E₂. In some embodiments, the threshold value, E₂, may equal 7.5.

Epitope extension process 816 may involve extending merged epitopes 814 to include additional residues in output subunits, which may allow for the generation of long, contiguous sequences to include in the resulting immunogen. A schematic illustrating the process of determining output extending merged epitopes 814 to determine output subunits 818 is shown in FIG. 1. In some embodiments, epitope extension process 816 may involve determining one or more residues that exist outside the combination of epitopes to include in output subunits 818. Epitope extension process 816 may involve computing a fitness cost associated with including the one or more residues in output subunits 818 may be computed and compared to a threshold value, E₃. In some embodiments, the threshold value, E₃, may equal 7. If the computed fitness cost is below the threshold value, then the one or more residues may be included in the output subunits 818. If the fitness cost exceeds the threshold value, then the one or more residues may be excluded from output subunits 818. According to some embodiments, epitope extension process 816 may involve determining one or more of merged epitopes 814 to exclude from the output subunits if the residue length of a merged epitope that has been subject to the extension process falls below a threshold length. For example, even after merging epitopes and extending the merged epitopes the resulting sequence regions are below a threshold length (e.g., 31 amino acids), then those sequence regions may be excluded from the output subunits 818 and not included in the resulting immunogen.

The threshold values used at the different steps of generating output subunits may vary, where a lower threshold corresponds to a more lenient inclusion criterion and a higher threshold corresponds to a more stringent inclusion criterion. The threshold values that are used may be guided by fitness penalties that correspond to the target virus being unable to evolve escape mutations over very long times. For Pol proteins, the specific threshold values used are E₁=8.5, E₂=7.5, and E₃=7.0. In the context of Pol proteins, a threshold may be used that is more stringent than for other proteins because it is not as immunogenic, and it may be desired to include only regions that contain residues where mutations are highly deleterious for virus fitness.

The threshold values for E₁, E₂, and E₃ associated with the steps of determining a combination of epitopes, merging the epitopes, and extending the merged epitopes, respectively, may vary. In some embodiments, E₁>E₂>E₃ to allow for more stringent inclusion criteria in implementing epitope combination technique 808 and more lenient inclusion criteria in implementing epitope merging process 812 and epitope extension process 816. If should be appreciated that other combinations of the threshold values may be implemented. For example, in some embodiments, the threshold values may be equal such that E₁=E₂=E₃. Yet, other embodiments may implement threshold values where E₁<E₂<E₃.

Some embodiments may involve generating a nucleotide sequence that encodes for the determined output subunits. As shown in FIG. 8, output subunits 818 may be analyzed using nucleotide sequence generation technique 820 to generate output nucleic acid sequence 822. In some embodiments, the vector may be an adenoviral vector. Other examples of suitable vectors that may be implemented to encode for immunogens designed using the techniques described herein are described above. In particular, some vectors may impose a constraint on the range of the total residue length of the immunogen to allow for efficient expression of the immunogen. For example, when the adenoviral vector is used for treatment, the total length of the construct may be between 300-1600 residues to allow for efficient expression of the construct. It should be appreciated that epitope merging process 812 and epitope extension process 816 may be repeated to include intervening amino acids. It should be appreciated that output nucleic acid sequence 822 may include the generated output subunits 818 in any suitable order. For example, it may be desired to vary junctional epitopes by shuffling the order of output subunits 818 as they appear in nucleic acid sequence 822.

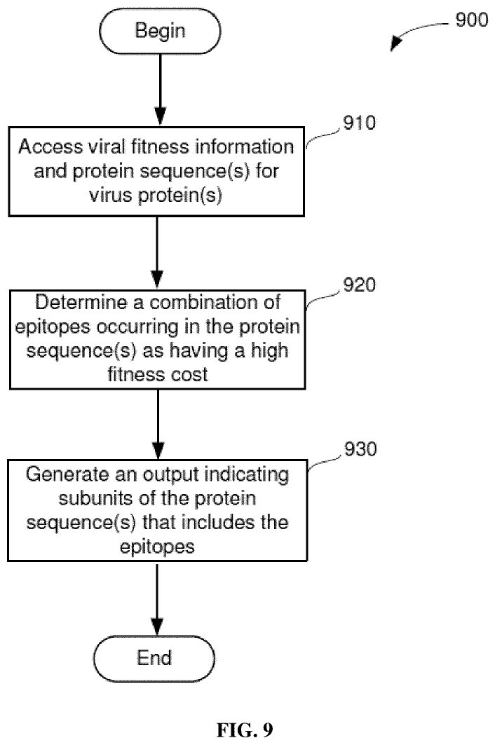

FIG. 9 is a flow chart of an illustrative process 900 for designing immunogens, in accordance with some embodiments of the technology described herein. Process 900 may be performed on any suitable computing device(s) (e.g., a single computing device, multiple computing devices co-located in a single physical location or located in multiple physical locations remote from one another, one or more computing devices part of a cloud computing system, etc.), as aspects of the technology described herein are not limited in this respect. In some embodiments, epitope combination technique 810, epitope merging process 812, and epitope extension process 816 may perform some or all of process 900 to design immunogens.

Process 900 begins at act 910, where viral fitness information associated with protein(s) of a virus and protein sequence(s) corresponding to the protein(s) are accessed. In some embodiments, the virus is HIV. Next, process 900 proceeds to act 920, where a combination of epitopes occurring in the protein sequence(s) as having a high fitness cost is determined by using the viral fitness information, such as by using epitope combination technique 810. In some embodiments, the combination of epitopes includes epitopes that account for coupling mutations of protein sequence(s). In some embodiments, the combination of epitopes includes one or more deleterious mutation regions of the protein sequence(s). In some embodiments, determining the combination of epitopes includes determining a first pair of epitopes as having a high fitness cost, comparing a fitness cost for a set of epitopes that includes the first pair and at least one other epitope to a first threshold value, and determining the combination of epitopes based at least in part of the comparing. In some embodiments, determining the combination of epitopes may involve including the first pair of epitopes and the at least one other epitope in the combination if the fitness cost is above the first threshold value. In some embodiments, determining the combination of epitopes further comprises including the first pair of epitopes in the combination if the fitness cost is below the first threshold value.

Next process 900 proceeds to act 930, where an output indicating subunits of the protein sequence(s) that have sequences of the epitopes in the combination are generated, such as by using epitope merging process 812, and epitope extension process 816. An indication of the output may be presented, such as to a user via a user interface. In some embodiments, generating the output indicating subunits may involve determining one or more residues of the protein(s) to include in the subunits that exists outside the combination of epitopes. In some embodiments, generating the output indicating subunits may involve determining one or more of the epitopes to exclude from the subunits.

In some embodiments, process 900 may further include an act of generating a polypeptide sequence for an immunogen having the combination of epitopes. In some embodiments, process 900 may further include an act of generating a nucleic acid sequence for a vector that encodes for the immunogen. In embodiments where the vector is an adenoviral vector, the immunogen may have a length between 300 and 1600 residues.

According to some embodiments, a process for designing immunogens according to the techniques described herein may include one or more of the following stages:

Seed: Begin the immunogen by finding the best pair of 11-mer epitopes with pairwise fitness cost greater than a threshold E₁. Selecting from the remaining epitopes in the protein, add the epitope with the highest average fitness cost when paired with the epitopes already in the immunogen. Repeat this selection and addition step until the average pairwise fitness cost of the new epitope, averaged over all pairs of epitopes in the immunogen, falls below E₁.

Bridge and merge: The output of stage 1 (Seed stage) is a list of subunits of variable length that are either non-contiguous or overlapping by <10 residues. (Because we assume putative epitopes are 11-mers, if two subunits overlapped by 10 residues, then they could be merged into one subunit without changing the included epitopes.) To bridge non-contiguous subunits, consider combinations of intervening amino acid segments between all successive subunits. Add a segment to the immunogen if the epitopes so included will not reduce the average pairwise fitness cost below a threshold E₂. To merge successive overlapping subunits, a similar procedure can be performed for the epitopes that would be included by combining the two subunits.

Extend or reject: Some of the subunits from stage 2 (Bridge and merge stage) may still be very short; when stitched together with other subunits, these would introduce more junctional epitopes than the number of natural epitopes that they contain. For these short subunits, consider all 31-mers that contain them. Include the best of these 31-mers in the immunogen as long as the average pairwise fitness cost of the new epitopes with the existing epitopes in the immunogen exceeds a threshold E₃. The subunits which cannot be extended this way due to poor synergy are removed from the immunogen.

Stages 2 and 3 can be repeated to include more intervening segments. Note that a lower threshold Eᵢ (i=1,2,3) corresponds to a more lenient inclusion criterion, whereas a higher threshold corresponds to a more stringent inclusion criterion. The threshold values that we used were guided by the fitness penalties that corresponded to the virus being unable to evolve escape mutations in patients for very long times. The specific values used for the thresholds are: E₁=8.5, E₂=7.5, and E₃=7.0 (for definition of E, see above equations). For Pol proteins, we use a threshold that is more stringent than for the other proteins (in particular, E_{i,Pol}=1.5E_{i,other} because it is not as immunogenic, and so we wish to include only the regions that contain residues where mutations are highly deleterious for virus fitness. Finally, the subunits in each immunogen can be concatenated in different orders: we designed the subunits both in their native 5′-to-3′ order as well as a shuffled variation, so that the potential junctional epitopes are varied.

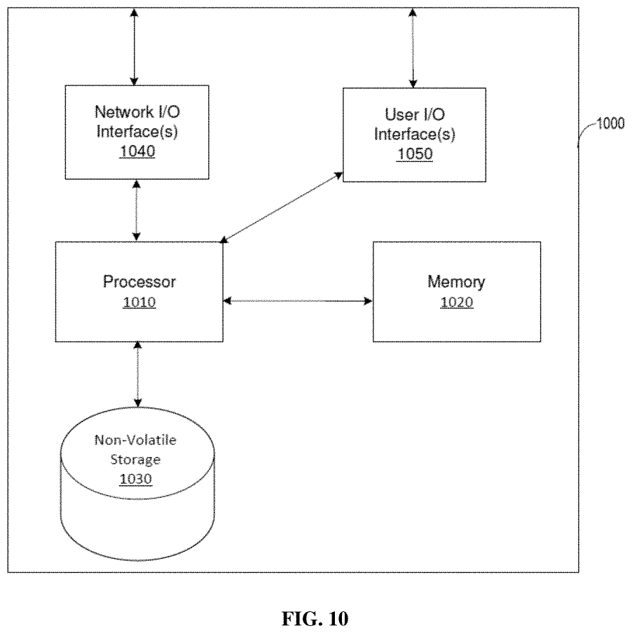

An illustrative implementation of a computer system 1000 that may be used in connection with any of the embodiments of the technology described herein is shown in FIG. 10. The computer system 1000 includes one or more processors 1010 and one or more articles of manufacture that comprise non-transitory computer-readable storage media (e.g., memory 1020 and one or more non-volatile storage media 1030). The processor 1010 may control writing data to and reading data from the memory 1020 and the non-volatile storage device 1030 in any suitable manner, as the aspects of the technology described herein are not limited in this respect. To perform any of the functionality described herein, the processor 1010 may execute one or more processor-executable instructions stored in one or more non-transitory computer-readable storage media (e.g., the memory 1020), which may serve as non-transitory computer-readable storage media storing processor-executable instructions for execution by the processor 1010.

Computing device 1000 may also include a network input/output (I/O) interface 1040 via which the computing device may communicate with other computing devices (e.g., over a network), and may also include one or more user I/O interfaces 1050, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

The terms “program” or “software” are used herein in a generic sense to refer to any type of computer code or set of processor-executable instructions that can be employed to program a computer or other processor to implement various aspects of embodiments as discussed above. Additionally, it should be appreciated that according to one aspect, one or more computer programs that when executed perform methods of the disclosure provided herein need not reside on a single computer or processor, but may be distributed in a modular fashion among different computers or processors to implement various aspects of the disclosure provided herein.

Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in one or more non-transitory computer-readable storage media in any suitable form. For simplicity of illustration, data structures may be shown to have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

Also, various inventive concepts may be embodied as one or more processes, of which examples have been provided. The acts performed as part of each process may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, and/or ordinary meanings of the defined terms.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In previous studies (see Barton et al., Nature Communications, 2016; Louie et al., PNAS, 2018; Goonetilleke and McMichael, Immunity, 2013, the relevant disclosures of each of which are herein incorporated by reference for the purpose and subject matter referenced herein), the “fitness landscape” of HIV proteins was defined. Herein, the fitness landscape was translated into knowledge of the intrinsic fitness of HIV proteins as a function of sequence, with explicit account for the effects of coupling between mutations. Subunits from the HIV-1 proteome having the highest fitness cost were selected using the algorithm disclosed herein and concatenated to make the immunogens of the present disclosure.

### Example 1

The two immunogens (nucleic acid sequences of unshuffled (SEQ ID NO:13) and shuffled (SEQ ID NO:14) forms shown in Table 2) were inserted into the E1 region of replication-defective Ad vectors from several serotypes (Ad26, RhAd66, etc) using standard methods (see Abbink et al. Journal of Virology, 2007; Abbink et al. Journal of Virology, 2018, the relevant disclosures of each of which are incorporated by reference herein for the purpose and subject matter referenced herein). Briefly, the Ad vectors were E1/E3 deleted, and the immunogens are inserted by recombination in the E1 position in E1-complementing cells. Vectors were then plaque purified, grown in complementing cells, and purified by CsCl density gradient sedimentation.

TABLE 2
Concatenated nucleotide sequence for two different versions
of an immunogen of the present disclosure.

| Version | Nucleotide sequence |
|---|---|
| immunogen: | ATGGTCTGGGCCAGCAGAGAGCTGGAAAGATTCGCCGTGAATCCCGGCCTGCT |
| 5-3 | GGAAACCTCTGAGGGCTGCAGACAGATCCTGGGACAGCTGCAGCAGGCCATCT |
| | CTCCCAGAACACTGAACGCCTGGGTCAAAGTGGTGGAAGAGAAGGCTTTCAGC |
| | CCCGAAGTGATCCCCATGTTCAGCGCCCTTTCTGAGGGCGCCACACCTCAGGA |
| | CCTGAACACCATGCTGAATACCGTTGGCGGACACCAGGCCGCCATGCAGATGC |
| | TGAAAGAGACAATCAACGAAGAGGCCGCCGAGTGGGATAGACTGCACCCTGTT |
| | CATGCCGGACCTATCGCTCCAGGCCAGATGAGAGAGCCTAGAGGCTCTGATAT |
| | CGCCGGCACCACCAGCACACTGCAAGAGCAGATCGGCTGGATGACCAACAATC |
| | CTCCTATTCCTGTGGGCGAGATCTACAAGCGGTGGATCATCCTGGGCCTGAAC |
| | AAGATCGTGCGGATGTACAGCCCCACCAGCATCCTGGATATCCGGCAGGGACC |
| | CAAAGAGCCCTTCAGAGACTACGTGGACCGGTTCTACAAGACCCTGAGAGCCG |
| | AGCAGGCCAGCCAAGAAGTGAAGAACTGGATGACAGAGACACTGCTGGTGCAG |
| | AACGCCAATCCTGACTGCAAGACCATCCTGAAGGCCCTGGGACCTGCCGCCAC |
| | ACTGGAAGAAATGATGACCGCCTGTCAAGGCGTTGGCGGCCCTGAAGCTTTGC |
| | TGGATACAGGCGCCGATGACACCGTGCTGGAAGAGATGAATCTGCCTGGCCGG |
| | TGGAAGCCCAAGATGATCGGAGGAATCGGCGGCTTCATCAAAGTGACCCCTGA |
| | CAAGAAGCACCAGAAAGAACCACCTTTCCTGTGGATGGGCTACGAGCTGCACC |
| | CCGATAAGTGGACCGTGCAGCCTATTGTGCTGCCCGAGAAGGATAGCTGGACC |
| | GTGAACGACATCCAGAAACTCGTGGGCAAGCTGAATTGGGCCAGCCAGATCTA |
| | CATGGAAAACCGGTGGCAAGTGATGATCGTGTGGCAGGTCGACCGGATGCGGA |
| | TCAGAACCTGGAAGTCCCTGGTCAAGCACCACATGTACATCGACGCCAAGCTG |
| | GTCATCACCACCTACTGGGGACTGCACACCGGCGAGAGAGATTGGCATCTTGG |
| | ACAGGGCGTGTCAATCGAGTGGCGGAAGTTCCTGGGCTTTCTGGGAGCCGCC |
| | GGATCTACAATGGGAGCTGCCAGCATCACCCTGACAGTGCAGGCTAGACAGCT |
| | GCTGAGCGGAATCGTGCAGCAGCAGAACAACCTGCTGAGAGCCATTGAGGCCC |
| | AGCAGCATCTCCTGCAGCTGACAGTGTGGGGCATCAAGCAGCTCCAGGCTAGA |
| | AGCCTGTGCCTGTTCAGCTACCACAGACTGAGGGACCTGCTGCTGATCGTGAC |
| | CCGGATTGTGGAACTGCTGGGAAGAAGAGGCTGGGAAGCCAATGCCGATTGCG |
| | CCTGGCTGGAAGCTCAAGAGGAAGAGGAAGTCGGCTTCCCCGTCAGACCTCAG |
| | GTGCCACTCAGACCCATGACCTACAAGTACAGCCAGAAGCGGCAGGACATCCT |
| | GGACCTGTGGGTGTACCACACACAGGGCTACTTCCCCGACTGGCAGAACTACA |
| | CACCTGGACCAGGC (SEQ ID NO: 13) |
| shuffled | ATGTACAGCCAGAAGCGGCAGGACATCCTGGACCTGTGGGTGTACCACACACA |
| immunogen | GGGCTACTTCCCCGACTGGCAGAACTACACACCTGGACCAGGACAGGCCATCT |
| | CTCCCAGAACACTGAACGCCTGGGTCAAAGTGGTGGAAGAGAAGGCTTTCAGC |
| | CCCGAAGTGATCCCCATGTTCAGCGCCCTTTCTGAGGGCGCCACACCTCAGGA |
| | CCTGAACACCATGCTGAATACCGTTGGCGGACACCAGGCCGCCATGCAGATGC |
| | TGAAAGAGACAATCAACGAAGAGGCCGCCGAGTGGGACAGACTGCATCCTGTT |
| | CATGCCGGACCTATCGCTCCCGGCCAGATGAGAGAACCTAGAGGCTCTGATAT |
| | CGCCGGCACCACCAGCACACTGCAAGAGCAGATCGGCTGGATGACCAACAATC |
| | CTCCTATTCCTGTGGGCGAGATCTACAAGCGGTGGATCATCCTGGGCCTGAAC |
| | AAGATCGTGCGGATGTACTCCCCTACCAGCATCCTGGATATCCGGCAGGGCCC |
| | CAAAGAGCCCTTCAGAGACTACGTGGACCGGTTCTACAAGACCCTGAGAGCCG |
| | AGCAGGCCAGCCAAGAAGTGAAGAACTGGATGACAGAGACACTGCTGGTGCAG |
| | AACGCCAATCCTGACTGCAAGACCATCCTGAAGGCCCTGGGACCTGCCGCCAC |
| | ACTGGAAGAAATGATGACCGCCTGTCAAGGCGTCGGCGGACCCACACCTGATA |
| | AGAAGCACCAGAAAGAACCACCGTTCCTGTGGATGGGCTACGAGCTGCACCCT |
| | GACAAGTGGACCGTGCAGCCTATTGTGCTGCCCGAGAAGGATAGCTGGACCGT |
| | GAACGACATCCAGAAACTCGTGGGCAAGCTGAACTGGGCCAGCCAGATCTACG |
| | ATGCCAAGCTGGTCATCACCACCTACTGGGGACTGCACACCGGCGAGAGAGAT |
| | TGGCATCTTGGACAGGGCGTGTCCATCGAGTGGCGGAAGTCCCTGTGCCTGTT |
| | CAGCTACCACAGACTGAGGGACCTGCTGCTGATCGTGACCCGGATTGTGGAAC |
| | TGCTGGGAAGAAGAGGCTGGGAAGCCGAGGCTCTGCTTGATACAGGCGCCGA |
| | TGATACCGTGCTGGAAGAGATGAACCTGCCTGGCAGATGGAAGCCCAAGATGA |
| | TCGGCGGCATCGGCGGATTCATCAAAGTCATGGAAAACCGGTGGCAAGTGATG |
| | ATCGTGTGGCAGGTCGACCGGATGCGGATCAGAACCTGGAAGTCTCTGGTCAA |
| | GCACCACATGTATATCTTTCTGGGATTCCTGGGCGCTGCCGGCTCTACAATGGG |
| | AGCCGCTTCTATCACCCTGACTGTGCAGGCTAGACAGCTGCTGAGCGGAATCG |
| | TGCAGCAGCAGAACAACCTGCTGAGAGCCATTGAGGCCCAGCAGCATCTCCTG |
| | CAGCTGACAGTGTGGGGCATCAAGCAGCTCCAGGCCAGAAATGCCGATTGCGC |
| | CTGGCTGGAAGCTCAAGAGGAAGAGGAAGTCGGCTTTCCCGTCAGACCTCAGG |
| | TGCCACTGAGGCCTATGACCTACAAAGTGTGGGCCAGCAGAGAGCTGGAAAGA |
| | TTCGCCGTGAATCCCGGCCTGCTGGAAACCTCTGAGGGCTGCAGACAGATCCT |
| | GGGGCAGCTGCAG (SEQ ID NO: 14) |

Four macaques were primed with the shuffled immunogen and boosted with the immunogen 5-3 from Table 3, and the immunogenicity of various peptide pools was measured using ELISPOT assay. FIGS. 7A and 7B include bar graphs showing the stimulation of the immune response in the macaques primed and after a later boost with the peptide immunogens.

```
TABLE 3
Concatenated amino acid sequence
for two different versions of
an immunogen of the
  present disclosure (the
initial M is not shown
  in these sequences but is covered
by this disclosure, and is
    encoded in the foregoing
  nucleic acid sequences).
```

| Version | Amino acid sequence |
|---|---|
| immunogen: | VWASRELERFAVNPGLLETSEGCRQI |
| 5-3 | LGQLQQAISPRTLNAWVKVVEEKAFSP |
| | EVIPMFSALSEGATPQDLNTMLNTVGG |
| | HQAAMQMLKETINEEAAEWDRLHPVHA |
| | GPIAPGQMREPRGSDIAGTTSTLQEQI |
| | GWMTNNPPIPVGEIYKRWIILGLNKIV |
| | RMYSPTSILDIRQGPKEPFRDYVDRFY |
| | KTLRAEQASQEVKNWMTETLLVQNANP |
| | DCKTILKALGPAATLEEMMTACQGVGG |
| | PEALLDTGADDTVLEEMNLPGRWKPKM |
| | IGGIGGFIKVTPDKKHQKEPPFLWMGY |
| | ELHPDKWTVQPIVLPEKDSWTVNDIQK |
| | LVGKLNWASQIYMENRWQVMIVWQVDR |
| | MRIRTWKSLVKHHMYIDAKLVITTYW |
| | GLHTGERDWHLGQGVSIEWRKFLGFLG |
| | AAGSTMGAASITLTVQARQLLSGIVQQ |
| | QNNLLRAIEAQQHLLQLTVWGIKQLQ |
| | ARSLCLFSYHRLRDLLLIVTRIVELLG |
| | RRGWEANADCAWLEAQEEEEVGFPVRP |
| | QVPLRPMTYKYSQKRQDILDLWVYHTQ |
| | GYFPDWQNYTPGPG |
| | (SEQ ID NO: 11) |
| shuffled | YSQKRQDILDLWVYHTQGYFPDWQNYT |
| immunogen | PGPGQAISPRTLNAWVKVVEEKAFSPE |
| | VIPMFSALSEGATPQDLNTMLNTVGGH |
| | QAAMQMLKETINEEAAEWDRLHPVHAG |
| | PIAPGQMREPRGSDIAGTTSTLQEQIG |
| | WMTNNPPIPVGEIYKRWIILGLNKIVR |
| | MYSPTSILDIRQGPKEPFRDYVDRFYK |
| | TLRAEQASQEVKNWMTETLLVQNANPD |
| | CKTILKALGPAATLEEMMTACQGVGGP |
| | TPDKKHQKEPPFLWMGYELHPDKWTVQ |
| | PIVLPEKDSWTVNDIQKLVGKLNWASQ |
| | IYDAKLVITTYWGLHTGERDWHLGQG |
| | VSIEWRKSLCLFSYHRLRDLLLIVTRI |
| | VELLGRRGWEAEALLDTGADDTVLEEM |
| | NLPGRWKPKMIGGIGGFIKVMENRWQV |
| | MIVWQVDRMRIRTWKSLVKHHMYIFL |
| | GFLGAAGSTMGAASITLTVQARQLLSG |
| | IVQQQNNLLRAIEAQQHLLQLTVWGI |
| | KQLQARNADCAWLEAQEEEEVGFPVRP |
| | QVPLRPMTYKVWASRELERFAVNPGLL |
| | ETSEGCRQILGQLQ |
| | (SEQ ID NO: 12) |

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A peptide immunogen comprising a plurality of HIV-1-specific immunogen subunits each having an amino acid sequence selected from the group consisting of SEQ ID NO: 1, 2, 3, 4, 5, 6, 7, 8, 9, and 10.

2. The peptide immunogen of claim 1, wherein the plurality of HIV-1 specific immunogen subunits is 5, 6, 7, 8, 9 or 10 HIV-1 specific immunogen subunits.

3. The peptide immunogen of claim 1, wherein the peptide immunogen comprises any order of 5 or more of the HIV-1-specific immunogen subunits.

4. The peptide immunogen of claim 1, wherein the peptide immunogen has an amino acid sequence of:
B₁B₂B₃B₄B₅B₆B₇B₈B₉B₁₀
wherein B₁, B₂, B₃, B₄, B₅, B₆, B₇, B₈, B₉, and B₁₀ are SEQ ID NOs: 1, 2, 3, 4, 5, 6, 7, 8, 9, and 10, respectively.

5. The peptide immunogen of claim 1, wherein the peptide immunogen has an amino acid sequence of SEQ ID NO:11 or SEQ ID NO:40.

6. The peptide immunogen of claim 1, wherein the peptide immunogen has an amino acid sequence of SEQ ID NO: 12 or SEQ ID NO:41.

7. The peptide immunogen of any one of claim 1, wherein the peptide immunogen has an amino acid sequence of:
B₁B₂B₃B₄B₆B₇B₈
wherein B₁, B₂, B₃, B₄, B₆, B₇, and B₈, are SEQ ID NOs: 1, 2, 3, 4, 6, 7, and 8, respectively.

8. The peptide immunogen of claim 1, wherein the amino acid sequence is SEQ ID NO: 34, SEQ ID NO:35, SEQ ID NO:42 or SEQ ID NO:43.

9. The peptide immunogen of claim 1, wherein conjugation of each HIV-1-specific immunogen subunit to another HIV-1 specific immunogen subunit creates a junctional epitope, wherein each junctional epitope is present once in the peptide immunogen.

10. The peptide immunogen of claim 1, wherein one or more of the HIV-1-specific immunogen subunits is repeated, optionally repeated once, provided that the repeated subunits are flanked by different subunits relative to each other, thereby creating different junctional epitopes at each repeated subunit.

11. The peptide immunogen of claim 1, wherein the length of the peptide immunogen ranges from 300 to 1,600 residues.

12. A nucleic acid comprising a nucleotide sequence that encodes any one of the peptide immunogens of claim 1.

13. The nucleic acid of claim 12, wherein the nucleotide sequence is SEQ ID NO: 13, SEQ ID NO:14, SEQ ID NO:36, SEQ ID NO:37, SEQ ID NO:38 or SEQ ID NO:39.

14.-24. (canceled)

25. A composition comprising the peptide immunogen of claim 1.

26.-29. (canceled)

30. A composition comprising the nucleic acid of claim 12.

31.-32 (canceled)

33. A method for treating a subject having or at risk of having an HIV-1 infection, comprising
administering to said subject an effective amount of the peptide immunogen of claim 1.

34.-40. (canceled)

41. A method for treating a subject having or at risk of having an HIV-1 infection, comprising
administering to said subject an effective amount of the nucleic acid of claim 12.

42.-53. (canceled)

54. A method, comprising:
accessing viral fitness information associated with one or more proteins of a virus and at least one protein sequence corresponding to the one or more proteins;
determining, using the viral fitness information, a combination of epitopes occurring in the at least one protein sequence as having a high fitness cost; and
generating an output indicating subunits of the at least one protein sequence that have sequences of the epitopes in the combination.

55.-65 (canceled)

66. A system comprising:
at least one hardware processor; and
at least one non-transitory computer-readable storage medium storing processor-executable instructions that, when executed by the at least one hardware processor, cause the at least one hardware processor to perform the method of claim 54.

67. At least one non-transitory computer-readable storage medium storing processor-executable instructions that, when executed by at least one hardware processor, cause the at least one hardware processor to perform the method of claim 54.
